(19) United States
(12) Patent Application Publication
(10) Pub. No.: US 2025/0339512 A1
(43) Pub. Date: Nov. 6, 2025

(54) SELF-CLEAVING POLYPROTEINS AND USES THEREOF

(71) Applicant: The University of Melbourne, Victoria (AU)

(72) Inventor: Joseph Torresi, Victoria (AU)

(73) Assignee: The University of Melbourne, Victoria (AU)

(21) Appl. No.: 18/294,900

(22) PCT Filed: Aug. 4, 2022
(86) PCT No.: PCT/AU2022/050844

§ 371 Date: Feb. 2, 2024

(30) Foreign Application Priority Data
Aug. 4, 2021 (AU) 2021902408

(51) Int. Cl. A61K 39/29; A61K 39/00; A61P 31/12; C12N 7/04; C12N 15/52; C12N 15/62

(52) U.S. Cl. CPC A61K 39/29; A61K 39/0011; A61P 31/12; C12N 7/04; C12N 15/52; C12N 15/62; A61K 2039/5258; C12N 2770/24223; C12N 2770/24252

(57) ABSTRACT

Disclosed herein are vaccine constructs for producing a virus-like particle (VLP) capable of raising an immune response to an immunogen, and uses thereof, wherein the constructs comprise nucleic acid sequences encoding an immunogen and a polyprotein, wherein the polyprotein comprises two or more viral structural proteins, wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP carrying the immunogen.

### INCORPORATION BY REFERENCE OF SEQUENCE LISTING

The Sequence Listing in an XML file, named as 43121_SequenceListing.xml of 48,000 bytes, created on Oct. 21, 2024, and submitted to the United States Patent and Trademark Office via Patent Center, is incorporated herein by reference.

### FIELD OF THE INVENTION

The present invention relates generally to vaccine constructs comprising nucleic acid sequences encoding self-cleaving polyproteins capable of self-assembling into viral-like particles (VLP) and uses thereof.

### BACKGROUND

As noted by Charlton Hume et al. (Biotechnol Bioeng. 2019; 116(4): 919-935), vaccination is one of the most effective ways of disease prevention and control. Indeed, viruses and bacteria that once caused catastrophic pandemics (e.g., smallpox, poliomyelitis, measles, and diphtheria) have either been eradicated or effectively controlled through vaccination programs.

Shaw and Feinberg (Clinical Immunology; Fourth Edition; 2013, pp1095-1121). reported that vaccines represent one of the most effective ways of disease prevention and control. Vaccination programs are currently estimated to save over 3 million lives each year, globally. In addition to its beneficial impact on vaccine-preventable disease morbidity and mortality, the direct and indirect impacts of vaccination programs translate into economic savings of many billions of dollars each year. Indeed, viruses and bacteria that once caused catastrophic pandemics (e.g., smallpox, poliomyelitis, measles, and diphtheria) have either been eradicated or effectively controlled through successful vaccination programs. These and other examples clearly highlight the benefits of vaccines in favourably manipulating host immunity to confer health benefits.

Current vaccine strategies will typically use live attenuated organisms, killed or inactivated organisms, subunit vaccines comprising purified (or partially purified) components of an organism, and subunit vaccines produced by recombinant DNA technologies. More recently, recombinantly-derived purified subunit vaccines have been developed that comprise virus-like particles (VLP), which exhibit immunoprotective traits of native viruses but are noninfectious. Several VLP that compositionally match a given natural virus have already been developed for clinical use. A plethora of studies also confirms that VLP can be designed to safely present heterologous antigens from a variety of pathogens and target antigens unrelated to the structural components of the VLP. Reference is made to U.S. Pat. Nos. 6,232,099 and 6,042,832, international patent applications WO 97/39134, WO 02/04007, WO 01/66778 and WO 02/00169, which provide illustrative examples of VLP carrying foreign peptides for immunotherapy.

Owing to this design versatility, VLP offer technological opportunities to modernise vaccine supply and disease response through rational bioengineering. These opportunities are enhanced with the application of synthetic biology, the redesign and construction of novel biological entities.

Therefore, whilst there have been significant advances in vaccine development, there still remains a critical need for improved vaccine compositions capable of inducing protective immunity against target proteins, including infectious agents (e.g., viruses, bacteria) and target antigens implicated in host pathologies (e.g., cancer associated antigens).

### SUMMARY

In an aspect disclosed herein, there is provided a vaccine construct comprising a nucleic acid sequence encoding a polyprotein, wherein the polyprotein comprises (i) an immunogen and (ii) two or more viral structural proteins capable of forming a virus-like particle (VLP), wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP.

In another aspect disclosed herein, there is provided a method of producing a VLP, the method comprising:
(i) introducing the vaccine construct described herein into a host cell; and
(ii) culturing the host cell of (i) under conditions and for a period of time sufficient for the host cell to produce the VLP.

In another aspect disclosed herein, there is provided a vaccine composition comprising (i) a first construct comprising a nucleic acid sequence encoding an immunogen and (ii) a second construct comprising a nucleic acid sequence encoding a polyprotein, wherein the polyprotein comprises two or more viral structural proteins capable of forming a virus-like particle (VLP), wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the immunogen and the liberated structural proteins to self-assemble into a VLP.

In another aspect disclosed herein, there is provided a method of producing a VLP, the method comprising:
(i) introducing the first and second vaccine constructs described herein into a host cell; and
(ii) culturing the host cell of (i) under conditions and for a period of time sufficient for the host cell to produce the VLP.

The present disclosure also extends to a VLP produced by the methods described herein, host cells and vaccine compositions comprising the vaccine constructs or the VLP described herein.

In another aspect disclosed herein, there is provided a method of raising an immune response in a subject to an immunogen, the method comprising administering to a subject in need thereof the vaccine construct, the VLP or the composition as described herein.

In another aspect disclosed herein, there is provided use of the vaccine construct, the VLP or the composition as described herein in the manufacture of a medicament for raising an immune response in a subject to the immunogen.

In another aspect disclosed herein, there is provided the vaccine construct, the VLP or the composition as described herein for use in raising an immune response in a subject against the immunogen.

Also disclosed herein is a kit comprising the vaccine construct, the VLP and/or the composition as described herein.

### BRIEF DESCRIPTION OF THE FIGURES

FIG. 1 is a polyprotein map of the multivalent HCV NS3 vaccine construct showing the HCV core, E1, E2 and NS3 proteins. A signal peptidase cleavage site is inserted between the (i) core and E1 proteins and (ii) E1 and NS3/E2 proteins.

FIG. 2 shows the NS3 nucleotide sequence from HCV1a clone H77. The forward and reverse primers used to generate the NS3 cDNA and confirm sequence integrity are shown in italics.

FIG. 3 shows the NS3 PCR amplification product (sitting at ˜1,893 kb).

FIG. 4 shows the polynucleotide and polypeptide sequences of the pAdTHCV1aCE1GFPE2 construct, including the position of the AflII and SpeI restriction sites and the location of the E1, E2, NS3 and M2 proteins.

FIG. 5 shows the purified NS3 PCR product and AflII/SpeI digested pAdHCV1aCE1E2 for ligation.

FIG. 6 shows the NS3 cDNA product following AflII/SpeI digestion of the pAdtrackCMVE1E2 plasmid.

FIG. 7 is an outline of the cloning strategy for the pAdTHCV1aCE1GFPE2 construct.

FIG. 8 provides the nucleotide sequence of the pAdTHCV1aCE1GFPE2 construct.

### DETAILED DESCRIPTION

Unless defined otherwise, all technical and scientific terms used herein have the same meanings as commonly understood by one of ordinary skill in the art to which the present disclosure belongs. Any materials and methods similar or equivalent to those described herein can be used to practice the present invention. Practitioners may refer to Sambrook et al., (1989) Molecular Cloning: A Laboratory Manual, 2nd ed., Cold Spring Harbor Press, Plainsview, N.Y., and Ausubel et al. (1999) Current Protocols in Molecular Biology (Supplement 47), John Wiley & Sons, New York, Murphy et al. (1995) Virus Taxonomy Springer Verlag: 79-87, for definitions and terms of the art and other methods known to the person skilled in the art.

The disclosure of every patent, patent application, and publication cited herein is hereby incorporated herein by reference in its entirety.

The citation of any reference herein should not be construed as an admission that such reference is available as “Prior Art” to the instant application.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

As used herein the singular forms “a”, “an” and “the” include plural aspects unless the context clearly dictates otherwise. Thus, for example, reference to “a cell” includes a single cell, as well as two or more cells; reference to “a VLP” includes one VLP, as well as two or more VLP; and so forth.

As used herein, the term “about” refers to approximately a +/−10% variation from a given value. It is to be understood that such a variation is always included in any given value provided herein, whether or not it is specifically referred to.

The present invention is predicated, at least in part, on the inventor's unique platform technology for generating recombinant, self-cleaving polyproteins capable of forming virus-like particles (VLP) that is adaptable to the production of any suitable VLP-based vaccine. Thus, in an aspect disclosed herein, there is provided a vaccine construct comprising a nucleic acid sequence encoding a polyprotein, wherein the polyprotein comprises (i) an immunogen and (ii) two or more viral structural proteins capable of forming a virus-like particle (VLP), wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP.

### Virus-Like Particles

Virus-like particles (VLP) have been shown to be useful as vaccines against a variety of infectious agents, including viral and bacterial infections. VLP are formed from the self-assembly of structural proteins of selected groups of viruses. These proteins self-assembly into a capsule, but, as none of the replicating nucleic acids are present, the VLP cannot replicate virus genome and create more or otherwise infectious virus particles. VLP are strictly non-infectious and generally harmless to the environment.

When VLP are formed in the presence of an immunogen, the VLP become delivery vehicles for the immunogen. VLP can also possess an antigenicity similar to the parent virus from which the structural components were obtained or derived and therefore useful as vaccines against that particular virus infection. VLP are generally useful as vaccines by possessing antigen within the components of the VLP. This allows for foreign or heterologous immunogens to be exposed on the surface of the VLP.

VLP are self-assembling complexes of capsid and/or envelope proteins (also referred to herein as viral structural proteins) that mimic the overall structure of their parental virus. VLP may also lack or possess dysfunctional copies of certain genes of the native virus, and this may result in the virus-like-particle being incapable of some function that is otherwise characteristic of the native virus, such as replication and/or cell-cell movement. Typically void of viral genetic material, VLP possess biologically desirable traits that are attributed, at least in part, to the particulate viral structure. Of particular interest is their efficient recognition, cellular uptake, and processing by host immune systems. VLP are also amenable to a broad range of modifications including encapsulation, chemical conjugation, and genetic manipulation (see, e.g., Roldão et al. Expert Rev Vaccines 2010; 9(10):1149-76). This versatility of VLP has prompted their use as suitable delivery agents for immunotherapy, noting that licensed prophylactic VLP vaccines such as Gardasil®, Cervarix®, Hecolin®, and Porcilis PCV® highlight VLP vaccines as being safe and effective. VLP also overcome some of the drawbacks associated with traditional vaccine production; namely, the infectious nature associated with live and inactivated vaccines and lengthy production time.

The term “self-assembly” typically refers to a process in which a system of pre-existing components, under specific conditions, adopts a more organised structure through interactions between the components themselves. In the present context, self-assembly refers to the intrinsic capacity of the viral structural proteins (e.g., capsid and/or envelope proteins) to self-assemble into VLP in the absence of other viral proteins, when subjected to specific conditions. “Self-assembly” does not preclude the possibility that cellular proteins such as chaperons participate in the process of intracellular VLP assembly. The self-assembly process may be influenced by factors such as choice of expression host, choice of expression conditions, and conditions for maturing the VLP. Virus capsid and/or envelope proteins may be able to form VLP on their own, or in combination with several virus capsid and/or envelope proteins, these optionally all being identical or related essential components of the virus structure.

The terms “virus-like particle” and “VLP” are therefore used interchangeably herein to refer to one or several recombinantly expressed viral structural (capsid and/or envelope) proteins, which spontaneously assemble into macromolecular particulate structures mimicking the morphology of a virus coat, but lacking infectious genetic material. As noted elsewhere herein, the polypeptide comprises, consists or consists essentially of at least two (e.g., 2, 3, 4, 5 and so on), preferably at least three, preferably at least four, more preferably at least five viral structural proteins. In another embodiment, the polypeptide comprises, consists or consists essentially of three viral structural proteins. In another embodiment, the polypeptide comprises, consists or consists essentially of four viral structural proteins. In another embodiment, the polypeptide comprises, consists or consists essentially of five viral structural proteins. In another embodiment, the polypeptide comprises, consists or consists essentially of six viral structural proteins. It is to be understood that the polypeptide may comprise any number of two or more viral structural proteins, including any combination of viral capsid and/or envelope proteins, as long as the two or more viral structural proteins, once liberated following host cell peptidase-dependent cleavage, are capable of self-assembly to form a VLP. It is to be understood that the term “self-assembly” refers to a process by which a system of pre-existing components, under specific conditions, adopts a more organised structure through interactions between the components themselves. In the present context, self-assembly refers to the intrinsic capacity of a viral structural (capsid and/or envelope) proteins to self-assemble into VLP in the absence of other viral proteins, when subjected to specific conditions. The term “self-assembly” is to be understood as not precluding the possibility that cellular proteins, such chaperons, participate in the process of intracellular VLP assembly. The self-assembly process may therefore be influenced by factors such as, but not limited to, choice of expression host, choice of expression conditions, and conditions for maturing the VLP. Virus capsid and/or envelope proteins may be able to form VLP on their own, or in combination with several virus capsid and/or envelope proteins.

Suitable VLP will be familiar to persons skilled in the art, illustrative examples of which include VLP created from virus or virus-like agents that infect humans, bacteria, parasites, fungus, plant, and/or other hosts. In this context, illustrative examples of suitable viruses from which VLP can be created include viruses of the family Flaviviridae, Coronaviridae, Orthomyxoviridae and Togaviridae. Thus, in an embodiment, the virus is of the family Flaviviridae, Coronaviridae, Orthomyxoviridae and Togaviridae.

### Flaviviridae

In an embodiment, the virus is of the family Flaviviridae. Suitable virus of the family Flaviviridae will be familiar to persons skilled in the art, illustrative examples of which include Flavivirus, Hepacivirus, Pegivirus and Pestivirus. Thus, in an embodiment disclosed herein, the virus is selected from the group consisting of a Flavivirus, a Hepacivirus, a Pegivirus and a Pestivirus.

In an embodiment, the virus is a Flavivirus (see, e.g., Laureti et al., Front. Immunol. 2018; 9:2180). Flavivirus virions are spherical, ˜50 nm in diameter, and consist of a nucleoprotein capsid enclosed in a lipid envelope. The RNA is a single 40S (˜10.9 kilobases) positive-sense strand and is capped at the 5′ end, but, unlike alphaviruses, has no poly A segment at the 3′ end. The virion has a single capsid protein (C) that is approximately 13,000 Da. The envelope consists of a lipid bilayer, a single envelope protein (E) of 51,000-59,000 Da, and a small nonglycosylated protein (M) of approximately 8,500 Da. Only E, which is glycosylated in most Flaviviruses, is clearly demonstrable on the virion surface. Flaviviruses can vary widely in their pathogenic potential and mechanisms for producing human disease. However, it is useful to consider them in three major categories: those associated primarily with the encephalitis syndrome (prototype: St. Louis encephalitis), with fever-arthralgia-rash (prototype: dengue fever), or with hemorrhagic fever (prototype: yellow fever). Classification within the genus Flavivirus is increasingly based upon antigenic relationships and genetic relationships. For example, Flaviviruses have been grouped into several antigenic complexes typified, by dissimilar viruses such as dengue, tick-borne encephalitis, St. Louis encephalitis, and yellow fever viruses. Flaviviruses are typically antigenically related by sharing common or similar antigenic determinants on C and E proteins. The single envelope glycoprotein, E, is the viral hemagglutinin; antibodies against E are involved in virus neutralization and haemagglutination inhibition. The antigenic determinants that induce neutralising antibody are specific, and species or subtypes of Flaviviruses are distinguished principally by neutralization tests. Haemagglutination inhibition tests reveal a broad range of cross-reactions among the Flaviviruses. Monoclonal antibody studies reveal genus, group, and virus-specific epitopes on the envelope glycoprotein. The nonstructural proteins also are antigenic, and at least one nonstructural protein, NS-1, contains both virus-specific and cross-reactive epitopes (see also Schmaljohn and McClain; Chapter 54: Alphaviruses (Togaviridae) and Flaviviruses (Flaviviridae); Medical Microbiology. 4th edition; Baron S, editor; Galveston (TX): University of Texas Medical Branch at Galveston; 1996)).

Suitable Flaviviruses will be familiar to persons skilled in the art, illustrative examples of which include Zika virus (see, e.g., Gorshkov et al. Front Microbiol. 2018; 9: 3252 and Laureti et al. ibid) and Dengue virus (see, e.g., Bäck and Lundkvist; Infect Ecol Epidemiol.; 2013; 3: 10.3402 and Laureti et al. ibid). In an embodiment disclosed herein, the Flavivirus is selected from the group consisting of a Zika virus and a Dengue virus

In an embodiment, the virus is a Hepacivirus. Suitable Hepaciviruses will be familiar to persons skilled in the art, illustrative examples of which include Hepatitis C virus (see, e.g., Li and Lo; World J Hepatol. 2015; 7(10): 1377-1389).

In an embodiment, the Hepacivirus is a Hepatitis C virus (HCV). HCV infects 2% of the world's population and is the leading cause of liver disease requiring transplantation. Recent advances in the treatment of HCV with directly acting antiviral agents (DAAs) have significantly improved sustained virological response rates. However, these treatments will not prevent re-infection particularly in high risk populations. Further, DAA therapies are still not affordable in most developing countries. With up to 90% of HCV cases occurring in injecting drug users (IDU) and as reinfection in this group is common, the expectation of controlling hepatitis C infection with antiviral drugs alone is not realistic. Simulation models of hepatitis C dynamics in high risk populations have predicted that the introduction of a vaccine will have a significant effect on reducing the incidence of hepatitis C. A vaccine with 50% to 80% efficacy targeted to high-risk IDU could dramatically reduce chronic HCV incidence in this population. Furthermore, vaccination after successful treatment with DAAs could also be as effective at reducing HCV prevalence as vaccinating an equivalent number of people who inject drugs (PWID) in the community. However, the limited access to treatment in the PWID and developing country populations means that a preventative HCV vaccine would ideally be capable of inducing an effective long lasting immune response in a single administration. However, at present a vaccine for HCV is not available. Most vaccine development strategies have typically focused on either the production of neutralising antibodies (Nab) or, alternatively, the induction of cell mediated immunity (CMI). A number of HCV vaccines that produce cellular immune responses against the core proteins have entered clinical trials. A meta-analysis of the efficacy of vaccine approaches in chimpanzees has also shown that immune responses to the structural proteins, especially the core protein, correlate closely with protection against and clearance of HCV. Mammalian cell derived genotype 1a and 3a HCV VLP's including core, E1 and E2 structural proteins have been described (Chua et al., PLoS One. 2012; 7(10):e47492, Collett et al., J Colloid Interface Sci, 2019; 545:259-268, and Kumar et al, Vaccine, 2016; 17; 34(8): 1115-25). These VLP have been shown to individually induce humoral and HCV-specific CD8+ T-cell responses. The generation and large-scale production of a VLP quadrivalent HCV vaccine comprising the structural proteins of HCV genotypes 1a, 1b, 2a and 3a has also been described (Earnest-Silveira et al., J Gen Virol. 2016; 97(8): 1865-1876).

An effective HCV vaccine will be required to generate cross-reactive CD4+, CD8+ T cell and/or neutralising antibody responses. In an embodiment, the VLP is a quadrivalent (genotype 1a/1b/2a/3a) HCV VLP. In preferred embodiments the HCV VLP is quadrivalent and can elicit both humoral and cellular immune responses from a single vaccine construct. The quadrivalent HCV VLP produces strong antibody responses, including broad neutralising antibodies, strong B and T cell responses against HCV in vaccinated mice and pigs (Christiansen et al., Viral Immunol, 2018, 31(4): 338-343, Christiansen et al. Sci Rep, 2018, 8(1): 6483, Christiansen et al. Sci Rep, 2019, 9(1): 9251 and Earnest-Silveira et al. Journal of virological methods, 2016, 236: 87-92.).

Currently known HCV types include HCV genotypes 1, 2, 3, 4, 5, 6, 7, 8, 9, 10, 11 and subtypes thereof include HCV subtypes 1a, 1b, 1c, 1d, 1e, 1f, 1g, 2a, 2b, 2c, 2d, 2e, 2f, 2g, 2h, 2i, 2k, 21, 3a, 3b, 3c, 3d, 3e, 3f, 3g, 4a, 4b, 4c, 4d, 4e, 4f, 4g, 4h, 4i, 4j, 4k, 4l, 4m, 5a, 6a, 6b, 7a, 7b, 7c, 7d, 8a, 8b, 8c, 8d, 9a, 9b, 9c, 10a and 11a. The sequences of cDNA clones covering the complete genome of several prototype isolates have been determined and include complete prototype genomes of the HCV genotypes 1a (e.g., GenBank accession number AF009606), 1b (e.g., GenBank accession number AB016785), 1c (e.g., GenBank accession number D14853), 2a (e.g., GenBank accession number AB047639), 2b (e.g., GenBank accession number AB030907), 2c (e.g., GenBank accession number D50409) 2k (e.g., GenBank accession number AB031663), 3a (e.g., GenBank accession number AF046866), 3b (e.g., GenBank accession number D49374), 4a (e.g., GenBank accession number Y11604), 5a (e.g., GenBank accession number AF064490), 6a (e.g., GenBank accession number Y12083), 6b (e.g., GenBank accession number D84262), 7b (e.g., GenBank accession number D84263), 8b (e.g., GenBank accession number D84264), 9a (e.g., GenBank accession number D84265), 10a (e.g., GenBank accession number D63821) and 11a (e g., GenBank accession number D63822). A further HCV genotype is described in International Patent Publication No. WO03/20970.

It will be understood by persons skilled in the art that structural proteins of HCV VLP, as herein described, may be derived from any HCV genotype known in the art. Typically, HCV genotypes 1a, 1b, 2a and 3a constitute the most common HCV genotypes globally. In an embodiment, the HCV core, E1 and E2 glycoproteins and/or NS protein of the HCV VLP, as described herein, are derived from a single HCV genotype selected from the group consisting of HCV genotypes 1a, 1b, 2a and 3a.

HCV viral structural proteins are generally known to include a core protein. E1 and E2 (70 kDa) glycoproteins. In some embodiments, the HCV polyprotein further comprises a non-structural (NS) viral protein. In an embodiment, the polyprotein comprises a HCV NS selected from the group consisting of NS proteins NS2, NS3, NS4A, NSSA, and NS5B. In the context of the present disclosure, the HCV proteins of the HCV VLP together form an HCV VLP which is capable of inducing an immune response against HCV.

The HCV “core protein” is a highly conserved basic protein which makes up the viral nucleocapsid. The core protein consists of HCV first 191 amino acids and can be divided into three domains on the basis of hydrophobicity. Domain 1 (amino acids 1-117) contains mainly basic residues with two short hydrophobic regions. Domain 2 (amino acids 118-174) is less basic and more hydrophobic and its C-terminus is at the end of p21. Domain 3 (amino acids 175-191) is highly hydrophobic and acts as a signal sequence for the HCV core protein. The term “HCV core protein” comprises the full-length HCV core protein, as well as functional fragments and derivatives thereof. In an embodiment, the full-length HCV core protein corresponds to the HCV polyprotein domain spanning amino acids 1-191 selected from the amino acid sequences of HCV genotype 1a (Genbank Accession No. AF009606), HCV genotype 1b (Genbank Accession No. AB016785), HCV genotype 2a (Genbank Accession No. AB047639) and HCV genotype 3a (Genbank Accession No. AF046866).

The HCV E1 and E2 glycoproteins are type I transmembrane proteins with a highly glycosylated N-terminal ectodomain and a C-terminal hydrophobic anchor viral structural proteins present on the viral membrane. After their synthesis, HCV glycoproteins E1 and E2 associate as a noncovalent heterodimer. Typically, the transmembrane domains of HCV envelope glycoproteins play a major role in E1/E2 heterodimer assembly and subcellular localization.

The term “HCV E1 glycoprotein” comprises the full-length HCV E1 glycoprotein, as well as functional fragments and derivatives thereof. In an embodiment, the full-length HCV E1 glycoprotein corresponds to the HCV polyprotein domain spanning amino acids 192-183 selected from the amino acid sequences of HCV genotype 1a (Genbank Accession No. AF009606), HCV genotype 1b (Genbank Accession No. AB016785), HCV genotype 2a (Genbank Accession No. AB047639) and HCV genotype 3a (Genbank Accession No. AF046866).

The term “HCV E2 glycoprotein” comprises the full-length HCV E2 glycoprotein, as well as functional fragments and derivatives thereof. In an embodiment, the full-length HCV E2 glycoprotein corresponds to the HCV polyprotein domain spanning amino acids 384-744 selected from the amino acid sequences of HCV genotype 1a (Genbank Accession No. AF009606), HCV genotype 1b (Genbank Accession No. AB016785), HCV genotype 2a (Genbank Accession No. AB047639) and HCV genotype 3a (Genbank Accession No. AF046866)

The HCV nonstructural (NS) proteins participate in virus assembly and include NS3, NS4A, NS4B, NS5A, and NS5B. The, term “HCV NS protein” comprises a full-length HCV NS protein, as well as functional fragments and derivatives thereof. In an embodiment, the full-length HCV NS protein corresponds to an HCV NS polyprotein domain derived from the amino acid sequences of HCV genotype 1a (Genbank Accession No. AF009606), HCV genotype 1b (Genbank Accession No. AB016785), HCV genotype 2a (Genbank Accession No. AB047639) and HCV genotype 3a (Genbank Accession No. AF046866).

In an embodiment, the NS protein of the HCV VLP is NS3. In an embodiment, the full-length HCV NS protein corresponds to the HCV polyprotein domain spanning amino acids 384-744 selected from the amino acid sequences of HCV genotype 1a (Genbank Accession No. AF009606), HCV genotype 1b (Genbank Accession No. AB016785), HCV genotype 2a (Genbank Accession No. AB047639) and HCV genotype 3a (Genbank Accession No. AF046866). In some embodiments, the HCV VLP comprises an NS protein from a different HCV genotype than the HCV genotype from which the HCV core, HCV E1 and/or HCV E2 glycoproteins are derived.

In an embodiment, the HCV VLP comprises a non-structural protein, such as NS3, to improve the breadth of CD8+ T cell responses. In certain embodiments, the modified HCV VLP will produce both HCV core and NS3 specific T cell responses, both important components for the prevention of HCV and important for the development of an effective vaccine for HCV. The insertion of HCV NS3 into the modified HCV VLP enables the production of broad cross protective neutralising antibody, CD4+ and CD8+ T cell responses.

In an embodiment, the two or more viral structural proteins are selected from the group consisting of an HCV core protein, an HCV envelope glycoprotein E1 and an HCV envelope glycoprotein E2. In an embodiment, the immunogen comprise an HCV NS3 protein. In an embodiment, the polyprotein comprises an HCV core protein, an HCV envelope glycoprotein E1, an HCV envelope glycoprotein E2 and an HCV NS3 protein.

In an embodiment, the HCV VLP is a tetravalent HCV VLP comprising a HCV core protein, a HCV envelope glycoprotein E1, a HCV envelope glycoprotein E2 and a NS3 protein.

In an embodiment, the Flavivirus is a Dengue virus. In an embodiment, the VLP is a Dengue VLP. In an embodiment, the Dengue VLP is a tetravalent DEN VLP. In an embodiment, the tetravalent DEN VLP comprises serotypes 1 2, 3 and 4.

In an embodiment, the two or more viral structural proteins are selected from the group consisting of a Dengue core (capsid) protein, a Dengue membrane (prM) protein and a Dengue envelope (E) protein. In an embodiment, the polyprotein comprises a Dengue core (capsid) protein, a Dengue membrane (prM) protein and a Dengue envelope (E) protein.

In an embodiment, the Flavivirus is a Zika virus. In an embodiment, the VLP is a Zika VLP.

### Coronaviridae

In another embodiment, the virus is of the family Coronaviridae (see Payne, S: Chapter 17—Family Coronaviridae; Viruses: From Understanding to Investigation, 2017, Pages 149-158; and Family—Coronaviridae: Virus Taxonomy, Ninth Report of the International Committee on Taxonomy of Viruses, 2012, Pages 806-828). The Coronaviridae family is typically divided into Coronavirinae and Torovirinae sub-families, which are further divided into six genera: Alphacoronavirus, Betacoronavirus, Gammacoronavirus, Deltacoronavirus, Torovirus, and Bafinivirus. While viruses in the genera Alphacoronaviruses and Betacoronaviruses infect mostly mammals, the Gammacoronavirus infect avian species and members of the Deltacoronavirus genus have been found in both mammalian and avian hosts (see, e.g., Phan et al., Virus Evol. 2018; 4(2): vey035). Coronaviruses (CoVs) cause a range of respiratory, enteric, and neurological diseases in human and animals. In human CoV infections, the severe acute respiratory syndrome coronavirus (SARS-CoV) and Middle East respiratory syndrome coronavirus (MERS-CoV) cause severe respiratory tract disease with high mortality rates, and there is strong evidence of zoonosis for both viruses. Given the zoonotic movement, detailed descriptions of the Coronaviridae in broad animal reservoirs that may cross the host barriers to cause diseases in humans are important.

Members of the Coronaviridae family share the same unique strategy for mRNA synthesis whereby the polymerase complex jumps or moves from one region of the template to a more distant region. The need for the polymerase complex to dissociate from the template may explain the high rate of RNA recombination that occurs during genome replication.

In the present context, suitable viruses of the family Coronaviridae will be familiar to persons skilled in the art, illustrative examples of which include Alphaletovirus (see, e.g., Bukhari et al.; Virology. 2018; 524:160-171) and Coronavirus (see, e.g., Fehr and Perlman; Coronaviruses. 2015; 1282: 1-23). Thus, in an embodiment disclosed herein, the virus is selected from the group consisting of Alphaletovirus and Coronavirus. In an embodiment disclosed herein, the Coronavirus is selected from the group consisting of Alphacoronavirus, Betacoronavirus, Deltacoronavirus and Gammacoronavirus. In an embodiment, the Coronavirus is Betacoronavirus. Suitable Betacoronaviruses will be familiar to persons skilled in the art, an illustrative example of which includes a Sarbecovirus. Thus, in an embodiment, the Betacoronavirus is a Sarbecovirus.

Suitable Sarbecoviruses will be familiar to persons skilled in the art, illustrative examples of which include Severe acute respiratory syndrome-related coronavirus, Severe acute respiratory syndrome coronavirus (SARS-CoV; see, e.g., Vijayanand et al., Clin Med (Lond). 2004; 4(2):152-60) and Severe acute respiratory syndrome coronavirus 2 (SARS-CoV-2; see, e.g., Khailany et al. Gene Rep. 2020; 19: 100682). In an embodiment disclosed herein, the Sarbecovirus is selected from the group consisting of Severe acute respiratory syndrome-related coronavirus, SARS-CoV and SARS-CoV-2. In an embodiment, the Sarbecovirus is SARS-CoV-2. As reported by Khailany et al. (ibid), the recent outbreak of coronavirus disease (COVID-19) that was first reported from Wuhan, China in December 2019. This epidemic spread to 220 countries and territories around the world, with over 123 million confirmed cases and over 2.7 million deaths. Similarly, Middle East respiratory syndrome coronavirus (MERS-CoV) had become a worldwide health concern. MERS-CoV originally reported in 2012, when it affected more than 2000 people in 27 countries and 4 sub-continents in the Middle East. SARS-CoV-2 is transmitted from person to person via droplet transmission and is therefore easily spread in overcrowded areas. Most patients experience only mild to moderate symptoms, such as high body temperature in conjunction with some respiratory symptoms such as cough, sore throat, and headache. Some people may have severe symptoms like pneumonia and acute respiratory distress syndrome. Notably, individuals with underlying complications such as heart disease, chronic lung disease, or diabetes potentially display more severe symptoms. To date, no specific antiviral treatment is proven effective, hence, infected people initially rely on symptomatic treatments that showed encouraging profile for blocking the new coronavirus in early clinical trials.

The genome SARS-CoV-2 varies from 29.8 kb to 29.9 kb and has a structure that is typical of other known coronaviruses, insofar as at the 5′ more than two-thirds of the genome comprises orflab encoding orflabpolyproteins, while at the 3′ one third consists of genes encoding structural proteins including surface (S), envelope (E), membrane (M), and nucleocapsid N proteins (see GenBank Accession No. NC_045512, the entire contents of which is incorporated herein by reference). Additionally, the SARS-CoV-2 contains 6 accessory proteins, encoded by ORF3a, ORF6, ORF7a, ORF7b, and ORF8.

In some contexts, it may be desirable to include two or more copies of the same viral structural proteins, as described herein. Without being bound by theory or by a particular mode of application, this approach may provide additional immunogen against which an immune response can be generated in a subject in which the vaccine construct or VLP is to be administered.

As disclosed elsewhere herein, the present inventor has shown that the nucleic acid sequences of the vaccine construct may be modified to incorporate heterologous peptide or protein sequences into the VLP. In some embodiments, this allows for the vaccine construct to be modified to produce a multivalent VLP comprising a plurality of immunogens against which an immune response can be generated. This advantageously allows a single VLP to be used to generate a protective immune response against a plurality of targets, including other viral and non-viral proteins, illustrative examples of which are described elsewhere herein.

The present disclosure also extends to VLP of viruses of the family Orthomyxoviridae, illustrative examples of which will be familiar to persons skilled in the art (see, e.g., Couch, Chapter 58: Orthomyxoviruses; Medical Microbiology. 4th edition; Baron S, editor; Galveston (TX): University of Texas Medical Branch at Galveston; 1996). In an embodiment, the Orthomyxoviridae virus is an influenza virus (see, e.g., Blut, Transfus Med Hemother. 2009; 36(1): 32-39; and Peteranderl et al., Semin Respir Crit Care Med. 2016; 37(4):487-500).

There are three subtypes of influenza viruses designated A, B, and C. The influenza virion contains a segmented negative-sense RNA genome. The influenza virion includes the following proteins: hemagglutinin (HA), neuraminidase (NA), matrix (M1), proton ion-channel protein (M2), nucleoprotein (NP), polymerase basic protein 1 (PB1), polymerase basic protein 2 (PB2), polymerase acidic protein (P A), and nonstructural protein 2 (NS2) proteins. The HA, NA, M1, and M2 are membrane associated, whereas NP, PB 1, PB2, P A, and NS2 are nucleocapsid associated proteins. The NS 1 is the only nonstructural protein not associated with virion particles but specific for influenza-infected cells. The M1 protein is the most abundant protein in influenza particles. The HA and NA proteins are envelope glycoproteins, responsible for virus attachment and penetration of the viral particles into the cell, and the sources of the major immunodominant epitopes for virus neutralization and protective immunity. Both HA and NA proteins are considered the most important components for prophylactic influenza vaccines.

Influenza virus infection is initiated by the attachment of the virion surface HA protein to a sialic acid-containing cellular receptor (glycoproteins and glycolipids). The NA protein mediates processing of the sialic acid receptor, and virus penetration into the cell depends on HA-dependent receptor-mediated endocytosis. In the acidic confines of internalized endosomes containing an influenza virion, the HA.sub.2 protein undergoes conformational changes that lead to fusion of viral and cell membranes and virus uncoating and M2-mediated release of M1 proteins from nucleocapsid-associated ribonucleoproteins (RNPs), which migrate into the cell nucleus for viral RNA synthesis. Antibodies to HA proteins prevent virus infection by neutralising virus infectivity, whereas antibodies to NA proteins mediate their effect on the early steps of viral replication.

Inactivated influenza A and B virus vaccines are licensed currently for parenteral administration. These trivalent vaccines are produced in the allantoic cavity of embryonated chick eggs, purified by rate zonal centrifugation or column chromatography, inactivated with formalin or β-propiolactone, and formulated as a blend of the two strains of type A and the type B strain of influenza viruses in circulation among the human population for a given year. The available commercial influenza vaccines are whole virus (WV) or subvirion (SV; split or purified surface antigen) virus vaccines. The WV vaccine contains intact, inactivated virions. SV vaccines treated with solvents such as tri-n-butyl phosphate (Flu-Shield, Wyeth-Lederle) contain nearly all of the viral structural proteins and some of the viral envelopes. SV vaccines solubilized with Triton X-100 (Fluzone, Connaught; Fluvirin, Evans) contain aggregates of HA monomers, NA, and NP principally, although residual amounts of other viral structural proteins are present. A potential cold-adapted live attenuated influenza virus vaccine (FluMist, MedImmune) was granted marketing approval recently by the FDA for commercial usage as an intranasally delivered vaccine indicated for active immunization and the prevention of disease caused by influenza A and B viruses in healthy children and adolescents, 5-17 years of age and healthy adults 18-49 years of age.

Several recombinant products have been developed as recombinant influenza vaccine candidates. These approaches have focused on the expression, production, and purification of influenza type A HA and NA proteins, including expression of these proteins using baculovirus infected insect cells and DNA vaccine constructs. Whilst these approaches have had some success, they remain ineffective in some patient groups.

The present disclosure also extends to VLP of viruses of the family Togaviridae, illustrative examples of which will be familiar to persons skilled in the art (see, e.g., Westaway et al., Intervirology. 1985; 24(3):125-39). In an embodiment, the virus of the family Togaviridae is an Alphavirus (see, e.g., Schmaljohn and McClain, ibidem). Alphavirus virions are typically spherical, 60 to 70 nm in diameter, with an icosahedral nucleocapsid enclosed in a lipid-protein envelope. Alphavirus RNA is a single 42S strand of approximately 4×10⁶ Da that is capped and polyadenylated. Alphavirus genomes that have been sequenced in their entirety are approximately 11.7 kb long. Virion RNA is positive sense, insofar as it can function intracellularly as mRNA, and the RNA alone has been shown experimentally to be infectious. The single capsid protein (C protein) has a molecular weight of around 30,000 Da. The alphavirus envelope consists of a lipid bilayer derived from the host cell plasma membrane and contains two viral glycoproteins (E1 and E2) of molecular weights of 48,000 to 52,000 Da. A small third protein (E3) of molecular weight 10,000 to 12,000 Da remains virion-associated in Semliki Forest virus but is dispatched as a soluble protein in most other alphaviruses. The proteins in the envelopes of alphaviruses are the viral glycoproteins, each anchored in the lipid at or near their C-terminus. On the virion surface, E1 and E2 are closely paired, and together form trimers that appear as “spikes” in an orderly array. Classification of alphaviruses is typically based upon antigenic relationships. Viruses have been grouped into seven antigenic complexes; typical species in four medically important antigenic complexes are Venezuelan equine encephalitis, eastern equine encephalitis, western equine encephalitis, and Semliki Forest viruses. Genome sequence information—typically obtained after viral RNA has been amplified by polymerase chain reaction (PCR)—is used with increasing frequency in the identification and classification of new viruses. The capsid protein induces antibodies, some of which are widely cross-reactive within the genus by complement fixation and fluorescent-antibody tests. Anti-capsid antibodies do not neutralise infectivity or inhibit hemagglutination. The E2 glycoprotein elicits and is thought to be the principal target of neutralising antibodies; however, some neutralising antibodies react with E1. Similarly, hemagglutination-inhibiting antibodies may react with either E2 or E1 Hemagglutination-inhibiting antibodies cross-react, sometimes extensively, among alphaviruses. Such cross-reactivity is attributable to the E1 glycoprotein, the amino acid sequences of which are more highly conserved among alphaviruses than those of E2. Neutralization assays are virus-specific, and species or subtypes are defined principally on the basis of neutralization tests.

In an embodiment, the VLP is suitable for treating a viral infection selected from the group consisting of influenza, yellow fever, hepatitis C, Nipah virus, Ross River and Chikungunya.

The present disclosure also extends to variants of the two or more viral structural proteins. The term “variant”, in this context, typically refers to a viral structural protein comprising an amino acid sequence having at least 80% sequence identity to the amino acid sequence of the native protein (i.e., as found in nature). Reference to “at least 80% sequence identity” includes 80%, 85%, 86%, 87%, 88%, 89%, 90%, 91%, 92%, 93%, 94%, 95%, 96%, 97%, 98%, 99% or 100% sequence identity to a native sequence, for example, after optimal alignment or best fit analysis. In an embodiment, the amino acid sequence of the structural protein has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to the corresponding protein found in nature, after optimal alignment or best fit analysis.

Where a variant of the structural protein is employed in the polyprotein disclosed herein, the variant will suitably be a “functional variant” (e.g., of the native sequence), as described elsewhere herein. It is to be understood that a “functional variant”, as used herein, means a peptide sequence that has a different amino acid sequence to the peptide to which it is compared (i.e., a comparator or reference sequence), including a natural (i.e., native) sequence or a synthetic variant thereof, yet retains the ability to self-assemble with the other structural protein(s) of the expressed polyprotein to form a VLP, as described herein. Suitable methods of determining whether a variant retains said function will be familiar to persons skilled in the art, illustrative examples of which are described elsewhere herein. A functional variant may include an amino acid sequence that differs from the reference sequence (e.g., a native sequence) by at least one (e.g., 1, 2, 3, 4 or 5) amino acid substitution, deletion or insertion, wherein said difference does not, or does not completely, abolish the ability of the variant to undergo host cell peptidase-depended cleavage. In some embodiments, the functional variant may comprise amino acid substitutions that enhance the ability of the variant to self-assemble with the other structural protein(s) of the expressed polyprotein to form a VLP, as described herein. In an embodiment, the functional variant differs from the native signal peptidase sequence by one or more conservative amino acid substitutions.

### Signal Peptidase Sequences

The terms “signal peptidase sequence” and “signalase sequence” are used interchangeably herein to mean an amino acid sequence that is specifically recognized and cleaved by peptidases. In the context of the present disclosure, the signal peptidase sequences are recognized and cleaved by host cell peptidases, also referred to herein as “signal peptide peptidases”. This advantageously allows the signal peptidase sequences to be cleaved by peptidases produced by the host cell. The peptidases may suitably be endogenous (i.e., native) to the host cell. Alternatively, or in addition, the host cell may be modified to produce one or more recombinant or heterologous peptidases capable of cleaving the signal peptidase sequence(s) of the polyprotein. Conveniently, the signal peptidase sequence(s) of the polyprotein will be capable of being cleaved by a peptidase that is endogenous to the host cell, thereby avoiding the need to modify the host cell to produce said peptidases. Thus, in an embodiment, the signal peptidase comprises a sequence that is capable of being cleaved by a peptidase that is native to the host cell. In other embodiments, the signal peptidase sequence(s) of the polyprotein will be capable of being cleaved by a peptidase that is heterologous to the host cell. In other embodiments, the polyprotein comprises (i) a signal peptidase sequence that is capable of being cleaved by a peptidase that is native to the host cell and (ii) a signal peptidase sequence that is capable of being cleaved by a peptidase that is heterologous to the host cell. In some embodiments, the polyprotein comprises a signal peptidase sequence that is native to the species from which the host cell is derived. In some embodiments, the polyprotein comprises a signal peptidase sequence that is heterologous to the species from which the host cell is derived.

The term “host cell peptidase-dependent cleavage” is to be understood to mean that the signal peptidase sequence is susceptible to cleavage by one or more peptidases (signal peptide peptidases) expressed by the host cell, whether the one or more peptidases are native to the host cell or recombinantly expressed by the host cell, as described elsewhere herein. In an embodiment, the signal peptidase sequence is susceptible to cleavage by one or more peptidases that are native to the host cell. Suitably, the signal peptidase sequence is not susceptible to virus peptidase-dependent or protease-dependent cleavage.

In an embodiment, the signal peptidase sequence is susceptible to host cell-dependent peptidases cleavage within the endoplasmic reticulum (ER) of the host cell. Suitable ER peptidases will be familiar to persons skilled in the art, illustrative examples of which are described in Oehler et al. (2012; J. Virol. 86(15):7818-7828), the entire contents of which are incorporated herein by reference.

It will be understood that the signal peptidase sequence will depend on the host cell peptidase to be employed to liberate the two or more viral structural proteins of the expressed polyprotein. Suitable signal peptidase sequences will be familiar to persons skilled in the art, illustrative examples of which include HCV core-E1 signal peptidase sequences, as described in Oehler et al. (2012; J. Virol. 86(15):7818-7828). In an embodiment, the signal peptidase sequence is a signal peptidase sequence utilized by hepatitis C virus, or a cleavable variant thereof. The term “cleavable variant” is to be understood to mean a variant (also referred to herein as a “functional variant” that has a different amino acid sequence to the peptide to which it is compared (i.e., a comparator or reference sequence), which may include a natural (i.e., native) sequence or a synthetic variant thereof, yet retains the ability to be cleaved by a host cell peptidase. In an embodiment, the signal peptidase sequence is selected from the group consisting of SEQ ID NOs:1-8 and amino acid sequences having at least 80% sequence identity to any of the foregoing.

### Core-E1 Signal Peptidase Sequences of Different HCV Genotypes

GT 1a
(SEQ ID NO: 1)
GCSFSIFLLALLSCLTVPASA
GT 1b
(SEQ ID NO: 2)
GCSFSIFLLALLSCLTIPASA
GT 2a
(SEQ ID NO: 3)
GFPFSIFLLALLSCITVPVSA
GT 3a
(SEQ ID NO: 4)
GCSFSVFLLALFSCLIHPAAS
GT 4a
(SEQ ID NO: 5)
GCSFSIFLLALLSCLTVPASA
GT 5a
(SEQ ID NO: 6)
GCSFSIFILALLSCLTVPTSA
GT 6a
(SEQ ID NO: 7)
GCSFSIFLLALLSCLTTPASA
GT 7a
(SEQ ID NO: 8)
GCSFSIFLLALLSCLTVPASA

In an embodiment, the signal peptidase sequence comprises, consists or consists essentially of the amino acid sequence of SEQ ID NO:1 or an amino acid sequence having at least 80% sequence identity thereto.

Reference to “at least 80% sequence identity” includes 80%, 85%, 86%, 87%, 88%, 89%, 90%, 91%, 92%, 93%, 94%, 95%, 96%, 97%, 98%, 99% or 100% sequence identity any one of SEQ ID NOs:1-8, for example, after optimal alignment or best fit analysis. In an embodiment, the signal peptidase sequence has at least 80%, preferably at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity to any one of SEQ ID NOs:1-8, for example, after optimal alignment or best fit analysis.

Where the signal peptidase sequence is a variant having an amino acid sequence having at least 80% sequence identity to any one of SEQ ID NO:1-8, the variant will suitably be a “functional variant” (e.g., of the native sequence). It is to be understood that a “functional variant”, as used herein, means a peptide sequence that has a different amino acid sequence to the peptide to which it is compared (i.e., a comparator or reference sequence), which may include a natural (i.e., native) sequence or a synthetic variant thereof, yet retains the ability to be cleaved by host cell peptidase, as described herein. Thus, in this context, the variant is also referred to herein as a cleavable variant.

Suitable methods of determining whether a variant retains said function will be familiar to persons skilled in the art, illustrative examples of which are described elsewhere herein. A functional variant may include an amino acid sequence that differs from the reference sequence (e.g., any one of SEQ ID NOs:1-8) by at least one (e.g., 1, 2, 3, 4 or 5) amino acid substitutions, deletions or insertions, wherein said difference does not, or does not completely, abolish the ability of the variant to undergo host cell peptidase-depended cleavage. In some embodiments, the functional variant may comprise amino acid substitutions that enhance the ability of the variant to undergo host cell peptidase-depended cleavage, as compared to the native signal peptidase sequence. In an embodiment, the functional variant differs from the native signal peptidase sequence by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

As used herein, the terms “identity”, “similarity”, “sequence identity”, “sequence similarity”, “homology”, “sequence homology” and the like mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. This may be referred to as conservative substitution. In an embodiment, the amino acid sequences may be modified by way of conservative substitution of any of the amino acid residues contained therein, such that the modification has no effect on the function of the modified polypeptide or polyprotein when compared to the unmodified polypeptide or polyprotein.

In some embodiments, sequence identity with respect to a peptide sequence relates to the percentage of amino acid residues in the candidate sequence which are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide or protein sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). Suitable programs for calculating the percent identity or similarity between sequences are generally known in the art.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, WI, USA) or by inspection and the best alignment (i.e., resulting in the highest percentage homology over the comparison window) generated by any of the various methods selected. Reference also may be made to the BLAST family of programs as for example disclosed by Altschul et al., 1997, Nucl. Acids Res.25:3389. A detailed discussion of sequence analysis can be found in Unit 19.3 of Ausubel et al., “Current Protocols in Molecular Biology”, John Wiley & Sons Inc, 1994-1998, Chapter 15.

In an embodiment, the signal peptidase sequence comprises, consists or consists essentially of an amino acid sequence selected from the group consisting of SEQ ID NOs:1-8.

The polyproteins encoded by the vaccine constructs disclosed herein will suitably comprise the two or more viral structural protein and the signal peptidase sequence(s) in a linear configuration, including as a fusion protein. As used herein, the term “fusion protein” typically refers to a polypeptide or polyprotein composed of two or more peptide sequences linked to one another. In an embodiment, the encoded polyprotein comprises two or more viral structural protein sequences linked to the signal peptidase sequence(s) end-to-end. In an embodiment, each of the two or more viral structural proteins of the polyprotein are separated by a signal peptidase sequence. Illustrative examples of suitable polyprotein configurations are described below (S=viral structural protein sequence and SP=signal peptidase sequence):
S1-SP1-S2
S1-SP1-S2-SP1-S3
S1-SP1-S2-SP2-S3
S1-SP1-S2-SP1-S3-SP1-S4
S1-SP1-S2-SP2-S3-SP2-S4
S1-SP1-S2-SP1-S3-SP2-S4
S1-SP1-S2-SP2-S3-SP3-S4

It is to be understood that any suitable configuration may be used, as long as two or more viral structural proteins of the polyprotein are separated by a signal peptidase sequence, as described herein.

In some embodiments, each of the two or more viral structural proteins of the polyprotein may be separated from one another by a signal peptidase sequence, as noted elsewhere herein and shown by the illustrative examples above. However, it is to be understood that the polyprotein may suitably comprises two or more viral structural proteins that are not separated by a signal peptidase sequence, but the polyprotein is otherwise still capable of generating a functional VLP following host cell peptidase-dependent cleavage of the polyprotein. Illustrative examples of suitable polyprotein configurations of this type, wherein at least two of the two or more viral structural proteins are separated by a signal peptidase sequence, are described below (S=viral structural protein sequence and SP=signal peptidase sequence):
S1-SP1-S2-S3
S1-S2-SP1-S3
S1-SP1-S2-S3-SP2-S4
S1-SP1-S2-S3-SP1-S4
S1-SP1-S2-SP2-S3-S4
S1-S2-SP1-S3-SP2-S4
S1-S2-SP1-S3-S4

In an embodiment, where the polypeptide comprises three viral structural proteins, each of the two or more viral structural proteins of the polyprotein may be separated from one another by a signal peptidase sequence of the polyprotein are separated by a signal peptidase sequence. In an embodiment, the two or more viral structural protein sequences are linked to one or two signal peptidase sequences via a suitable linking moiety, also referred to herein as a linker. Suitable methods of linking peptide sequences will be familiar to persons skilled in the art, illustrative examples of which include peptide (amide) bonds and linkers. As used herein, the term “linker” refers to a short polypeptide sequence interposed between any two neighboring peptide or protein sequences as herein described. In an embodiment, the linker is a polypeptide linker of 1 to 10 amino acids, preferably 1, 2, 3, 4 or 5 naturally or non-naturally occurring amino acids. In an embodiment, the linker is a carbohydrate linker. Suitable carbohydrate linkers will be known to persons skilled in the art. In another embodiment disclosed herein, the fusion protein comprises one or more peptidic or polypeptidic linker(s) together with one or more other non-peptidic or non-polypeptidic linker(s). Further, different types of linkers, peptidic or non-peptidic, may be incorporated in the same fusion peptide as deemed appropriate. In the event that a peptidic or polypeptidic linker is used to join two respective peptide sequences, the linker will be advantageously incorporated such that its N-terminal end is bound via a peptide bond to the C-terminal end of the one peptide sequence, and its C-terminal end via a peptide bond to the N-terminal end of the other peptide sequence. The individual peptide sequences within the fusion protein may also have one or more amino acids added to either or both ends, preferably to the C-terminal end. Thus, for example, linker or spacer amino acids may be added to the N- or C-terminus of the peptides or both, to link the peptides and to allow for convenient coupling of the peptides to each other and/or to a delivery system such as a carrier molecule serving as an anchor. An illustrative example of a suitable peptidic linker is LP (leucine-proline). The immunogen may suitably comprise a fusion protein comprising, consisting, or consisting essentially of any combination of two or more of the peptide sequences. In an embodiment, the fusion protein comprises, consists, or consists essentially of at least three of the peptide sequences. Also contemplated herein are fusion proteins comprising at least two of the peptide sequences disclosed herein, concatenated two or more times in tandem repeat.

### Immunogens

The term “immunogen”, as used herein, typically refers to a molecule, molecules, a portion or portions thereof, or a combination of molecules, including whole cells and tissues, which are capable of inducing an immune response in a subject. The immunogen may suitably comprise a single epitope or it may comprise a plurality of epitopes, including B cell and T cell epitopes or mimotopes thereof. Immunogens may therefore encompass peptides, carbohydrates, proteins, nucleic acids, and various microorganisms, in whole or in part, including viruses, bacteria and parasites. Antigens and haptens are also encompassed by the term “immunogen”, as used herein.

An immunogen is typically capable of raising an immune response, including a humoral (antibody) and/or cellular immune response, in vivo, whether alone or when combined (e.g., co-administered to a subject) with a suitable adjuvant. The terms “peptide” and “polypeptide” are used interchangeably herein in their broadest sense to refer to a molecule of two or more amino acid residues, or amino acid analogs. The amino acid residues may be linked by peptide bonds, or alternatively by other bonds, e.g. ester, ether etc., but in most cases will be linked by peptide bonds. The terms “amino acid” or “amino acid residue” are used herein to encompass both natural and unnatural or synthetic amino acids, including both the D- or L-forms, and amino acid analogs. An “amino acid analog” is to be understood as a non-naturally occurring amino acid differing from its corresponding naturally occurring amino acid at one or more atoms. For example, an amino acid analog of cysteine may be homocysteine.

The polyprotein encoded by the vaccine construct will suitably comprise at least one immunogen. By “at least one immunogen” is meant 1, 2, 3, 4, 5, 6, 7, 8, 9, 10, 11 or more peptide sequences capable of raising a humoral (antibody) and/or cellular immune response in vivo when administered to an immunocompetent subject. In an embodiment, the polypeptide or polyprotein encoded by the vaccine construct comprises at least 1, preferably at least 2, preferably at least 3, preferably at least 4, preferably at least 5, preferably at least 6, preferably at least 7, preferably at least 8, or more preferably at least 9 immunogens. In an embodiment, each of the at least one immunogen is capable of raising a humoral (antibody) and/or cellular immune response to a different target antigen in vivo. In another embodiment, where the polypeptide or polyprotein encoded by the vaccine construct comprises a plurality of immunogens, 2 or more of the plurality of immunogens are capable of raising a humoral and/or cellular immune response to the same target antigen in vivo. For instance, each immunogen may be capable of raising a humoral and/or cellular immune response directed to a different epitope of the target antigen in vivo. Advantageously, the at least one immunogen is capable of raising a humoral and/or cellular immune response to an extracellular portion of the native (e.g., wild-type) target antigen in vivo.

Where the polyprotein encoded by the vaccine construct comprises at least two immunogens, the at least two immunogens may have a branched or linear configuration, including as a fusion protein. As used herein, the term “fusion protein” typically refers to a polypeptide or polyprotein composed of two or more peptide or protein sequences linked to one another. In an embodiment, the fusion protein comprises two or more peptide or protein sequences linked to one another end-to-end. In an embodiment, the fusion protein comprises two or more peptide or protein sequences linked to one another in a linear configuration via a suitable linking moiety, also referred to herein as a linker. Suitable methods of linking peptide sequences will be familiar to persons skilled in the art, illustrative examples of which include peptide (amide) bonds and linkers. As used herein, the term “linker” refers to a short polypeptide sequence interposed between any two neighboring peptide sequences as herein described. In an embodiment, the linker is a polypeptide linker of 1 to 10 amino acids, preferably 1, 2, 3, 4 or 5 naturally or non-naturally occurring amino acids. In an embodiment, the linker is a carbohydrate linker. Suitable carbohydrate linkers will be known to persons skilled in the art. In another embodiment disclosed herein, the fusion protein comprises one or more peptidic or polypeptidic linker(s) together with one or more other non-peptidic or non-polypeptidic linker(s). Further, different types of linkers, peptidic or non-peptidic, may be incorporated in the same fusion peptide as deemed appropriate. In the event that a peptidic or polypeptidic linker is used to join two respective peptide sequences, the linker will be advantageously incorporated such that its N-terminal end is bound via a peptide bond to the C-terminal end of the one peptide sequence, and its C-terminal end via a peptide bond to the N-terminal end of the other peptide sequence. The individual peptide sequences within the fusion protein may also have one or more amino acids added to either or both ends, preferably to the C-terminal end. Thus, for example, linker or spacer amino acids may be added to the N- or C-terminus of the peptides or both, to link the peptides and to allow for convenient coupling of the peptides to each other and/or to a delivery system such as a carrier molecule serving as an anchor. An illustrative example of a suitable peptidic linker is LP (leucine-proline). The immunogen may suitably comprise a fusion protein comprising, consisting, or consisting essentially of any combination of two or more of the peptide sequences. In an embodiment, the fusion protein comprises, consists, or consists essentially of at least three of the peptide sequences. Also contemplated herein are fusion proteins comprising at least two of the peptide sequences disclosed herein, concatenated two or more times in tandem repeat.

Without being bound by theory or by a particular mode of application, it will be understood that incorporating two or more different immunogens into the VLP, as herein described, may suitably generate a more beneficial immune response by eliciting a higher antibody titre or enhanced immune cell activation as compared to a VLP comprising a single immunogen.

It is to be understood that the immunogen, as described herein, may comprise an amino acid sequence of any suitable length, as long as the immunogen retains the ability or capacity to induce an immune response in vivo to the target antigen.

As used herein, the term “B cell epitope” refers to a part of a molecule that is recognized by an antibody. Thus, a “B cell epitope” is to be understood as being a smaller subsequence of an antigen that is capable of being recognized (bound) by an antibody. It is to be understood that an antigen may contain multiple B cell epitopes, and therefore may be bound by multiple distinct antibodies. A single epitope may also be bound by multiple antibodies having different antigen-binding specificity and/or affinity. Multiple antibodies of different subclasses may also bind to the same epitope.

In an embodiment, the immunogen will comprise at least one peptide sequence or protein that, when administered to a subject, will induce an antibody response such that the antibody binds to a B cell epitope of the target antigen, preferably to a B cell epitope of a native target antigen; that is, to the target antigen as it exists in nature. Preferably, the at least one peptide or protein sequence of the immunogen will induce an antibody response such that the antibody binds to a B cell epitope of the extracellular domain of a native target antigen. Preferably, the B cell epitope to which an antibody is raised will be an epitope located within the extracellular domain of the native target antigen. In an embodiment, the at least one immunogen comprises an autologous B cell epitope of the target antigen; that is, a B cell epitope of the target antigen having an amino acid sequence derived from a target antigen of the same species as the subject to be treated.

As used herein, the term “T cell epitope” means an epitope presented on the surface of an antigen-presenting cell bound to an MHC molecule. T cell epitopes presented by MHC class I molecules are typically peptides between 8 and 11 amino acids in length, whereas MHC class II molecules are present as longer peptides, typically 13-17 amino acids in length.

In an embodiment, the immunogen will comprise at least one peptide sequence that, when administered to a subject, will induce a T cell response towards the target antigen, preferably to a T cell epitope of a native target antigen; that is, to the target antigen as it exists in nature. B cell and T cell epitopes of target antigens can be identified by persons skilled in the art using known methodologies, illustrative examples of which are described in Sanchez-Trincado et al. (Journal of Immunology Research, 2017; article ID 2680160).

As used herein, the terms “native” and “natural” refer to the form of a molecule as normally occurring in nature. Conversely, a “non-native” sequence, including a “non-native linker” is any amino acid sequence not belonging to native sequence of the target antigen.

The present disclosure also extends to immunogens capable of raising a humoral and/or cellular immune response against isoforms of non-human species, including non-human primate, canine, feline, equine, bovine, porcine and murine isoforms of the target antigen.

The present disclosure also extends to the use of immunogens that are a “functional variant” of the native target sequence. It is to be understood that a “functional variant”, as used herein, means a peptide sequence that has a different amino acid sequence to a peptide to which it is compared (i.e., a comparator), which may include a natural (i.e., native) sequence or a synthetic variant thereof, yet retains the ability to induce a humoral and/or cellular immune response in vivo to the target antigen.

Suitable methods of determining whether a functional variant retains the ability to induce a humoral and/or cellular immune response in vivo against the native target antigen will be familiar to persons skilled in the art, illustrative examples of which are described elsewhere herein. A functional variant may include an amino acid sequence that differs from the native peptide sequence by one or more (e.g., 1, 2, 3, 4, 5, 6, 7, 8, 9 or more) amino acid substitutions, wherein said difference does not, or does not completely, abolish the capacity of the variant to induce a humoral and/or cellular immune response towards the target antigen. In some embodiments, the functional variant may comprise amino acid substitutions that enhance the capacity of the peptide sequence to induce a humoral and/or cellular immune response to the target antigen, as compared to the native peptide sequence. In an embodiment, the functional variant differs from the native peptide sequence by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

In an embodiment, a functional variant includes amino acid substitutions and/or other modifications in order to increase the stability of the immunogen and/or to increase the solubility of the immunogen to enhance its ability to induce an antibody response in vivo. Suitable modifications will be familiar to persons skilled in the art, illustrative examples of which are described elsewhere herein.

In an embodiment, the immunogen comprises a promiscuous T helper (Th) cell epitope. Suitable promiscuous Th cell epitopes will be known to persons skilled in the art, illustrative examples of which include measles virus fusion protein (MVF; KLLSLIKGVIVHRLEGVE; SEQ ID NO:9), tetanus toxoid (TT; NSVDDALINSTIYSYFPSV; SEQ ID NO:10), TT1 (PGINGKAIHLVNNQSSE; SEQ ID NO:11); TT peptide P2 (QYIKANSKFIGITEL; SEQ ID NO:12); TT peptide P30 (FNNFTVSFWLRVPKVSASHLE; SEQ ID NO:13); MVF′ (LSEIKGVIVHRLEGV; SEQ ID NO:14); Hepatitis B virus (HBV; FFLLTRILTIPQSLN; SEQ ID NO:15); circumsporozoite protein (CSP; TCGVGVRVRSRVNAANKKPEl; SEQ ID NO:16), and those described in WO2000/046390, the entire contents of which is incorporated herein by reference. In an embodiment, the promiscuous Th epitope is from about 8 to about 36, preferably from about 8 to about 24, more preferably from about 8 to 22, most preferably from about 8 to about 22 amino acids in length. In an embodiment, the promiscuous Th cell epitope is linked to a peptide sequences or fusion protein, as herein described, to form a chimeric peptide. Suitable linkers will be familiar to persons skilled in the art, illustrative examples of which are described elsewhere herein.

As used herein, the term “B cell epitope” refers to a part of a molecule that is recognized by an antibody. Thus, a “B cell epitope” is to be understood as being a smaller subsequence of an antigen that is capable of being recognized (bound) by an antibody. It is to be understood that an antigen may contain multiple B cell epitopes, and therefore may be bound by multiple distinct antibodies. A single epitope may also be bound by multiple antibodies having different antigen-binding specificity and/or affinity. Multiple antibodies of different subclasses may also bind to the same epitope.

In an embodiment, the immunogen comprises at least one peptide sequence that, when administered to a subject, will induce an antibody response such that the antibody binds to a B cell epitope of a native target antigen; that is, to the target antigen as it exists in nature. Preferably, the at least one peptide sequence of the immunogen will induce an antibody response such that the antibody binds to a B cell epitope of the extracellular domain of a native target antigen. Preferably, the B cell epitope to which an antibody is raised will be an epitope located within the extracellular domain of the native antigen. In an embodiment, the immunogen comprises an autologous B cell epitope of the target antigen.

Methods of determining the B cell epitope of a target antigen will be familiar to persons skilled in the art. In an illustrative example, a sub-sequence of an antigen may be identified with a high degree of accuracy as being, or comprising, a B cell epitope by using established computer programs that compare the subsequence in question with a database of known sequences and/or partial sequences known to be recognized by antibodies encoded by the human or mouse germline. Alternatively, a B cell epitope may be identified by computer-aided analysis using various combinations of correlates of antigenicity such as surface accessibility, chain flexibility, hydropathy/hydrophilicity profiles, predicted secondary structure, etc. Alternatively, an antigen may be identified as comprising a B cell epitope by immunising an animal with the antigen in question at least once, allowing a humoral immune response to mount and then testing the serum of the animal for antibodies that specifically bind to at least a part of the administered antigen using, for example, an enzyme linked immunosorbant assay (ELISA), a radioimmunoassay, a Western blot analysis or a dot-blot analysis.

As noted elsewhere herein, the immunogen will comprise at least one peptide sequence that is capable of inducing a humoral and/or cellular immune response towards the native target antigen. This may be achieved by using a peptide sequence comprising an amino acid sequence of a B cell epitope and/or a T cell epitope of the target antigen (or a functional variant thereof), and/or a mimotope thereof. As used herein, the term “mimotope” refers to a molecule that has a conformation that has a topology equivalent to the B cell epitope or the T cell epitope of which it is a mimic such that it is capable of raising a humoral and/or cellular immune response that targets the same epitope of the native target antigen. In other words, when administered to a host, the VLP comprising the mimotope will elicit an immune response (humoral and/or cellular) in a host towards the target antigen of which it is a mimic.

Peptide sequences disclosed herein can be synthetically produced by chemical synthesis methods which are well known in the art, either as an isolated peptide sequence or as a part of another peptide or polypeptide. Alternatively, peptide or protein sequences can be produced in a microorganism which produces the (recombinant) peptide or protein sequence or sequences, which can then be isolated and, if desired, further purified. The peptide or protein sequences can be produced in microorganisms such as bacteria, yeast or fungi, in eukaryote cells such as a mammalian or an insect cell, or in a recombinant virus vector such as adenovirus, poxvirus, herpesvirus, Semliki forest virus, baculovirus, bacteriophage, sindbis virus or sendai virus. Suitable bacteria for producing the peptide or protein sequences will be familiar to persons skilled in the art, illustrative examples of which include E. coli, B. subtilis or any other bacterium that is capable of expressing the peptide sequences. Illustrative examples of suitable yeast types for expressing the peptide or protein sequences include Saccharomyces cerevisiae, Schizosaccharomyces pombe, Candida, Pichia pastoris or any other yeast capable of expressing peptides. Corresponding methods are well known in the art. Also methods for isolating and purifying recombinantly produced peptide sequences are well known in the art and include, for example, gel filtration, affinity chromatography and ion exchange chromatography.

As noted elsewhere herein, the term “immunogen” refers to a molecule, molecules, a portion or portions thereof, or a combination of molecules, including whole cells and tissues, which are capable of inducing an immune response in a subject. The immunogen may suitably comprise a single epitope or it may comprise a plurality of epitopes, including B cell and T cell epitopes or mimotopes thereof. Immunogens may therefore encompasses peptides, carbohydrates, proteins, nucleic acids, and various microorganisms, in whole or in part, including viruses, bacteria and parasites. Antigens and haptens are also encompassed by the term “immunogen”, as used herein.

The immunogen will typically comprise an antigen or other structure (e.g., a mimotope) that stimulates an immune response in an individual. Suitable immunogens will be familiar to persons skilled in the art and will largely depend on the disease or condition to be treated or prevented. Illustrative examples include peptides, proteins, lipids, fatty acids, polysaccharides, lipopolysaccharides. Typically, the immunogen will be presented on the outer surface of the VLP. Immunogens may be specific for a particular infectious agent or combination of agents such as, for example, viral infectious agents; for example, enterovirus, hepatitis virus, human immunodeficiency virus (HIV), human papilloma virus (HPV), influenza virus, pertussis virus, rubella virus, tetanus, varicella virus (VZV), flavivirus, West Nile virus, dengue virus, tick-borne encephalitis virus, yellow fever virus, Zika virus), bacterial infectious agents (e.g., Chlamydia, Clostridium, diphtheria, meningococcal, streptococcal, staphylococcal, pneumococcal), parasitic infectious agents (e.g., giardia, malaria (plasmodium)), an agent that causes sepsis or septicaemia, and the viruses disclosed elsewhere herein.

In some embodiments, the immunogen is a virus antigen that is native to the VLP, or a mimotope thereof. For example, if the VLP is a HCV VLP, the immunogen may be selected from the group consisting of a HCV core protein, a HCV envelope glycoprotein E1, a HCV envelope glycoprotein E2, a NS3 protein and a combination of one or more of the foregoing. Other illustrative examples of virus antigens include influenza matrix protein peptide 58-66 (Jager et al., Int. J. Cancer 67: 54 (1996)), HPV16 E7 peptide 86-93 (van Driel et al., Eur. J. Cancer 35:946 (1999)), HPV E7 peptide 12-20 (Scheibenbogen C et al., J. Immunother 23: 275 (2000)), HPV16 E7 peptide 11-20 (Smith et al., J. Clin. Oncol. 21: 1562 (2003)), HSV2 gD (Berman et al., Science 227: 1490 (1985)), CMV gB (Frey et al., Infect Dis. 180: 1700 (1999), Gonczol et al., Exp. Opin. Biol. Ther. 1: 401 (2001)), and CMV pp 65 (Rosa et al., Blood 100: 3681 (2002); and Gonczol et al., Exp. Opin. Biol. Ther. 1: 401 (2001)).

Alternatively, or in addition, the immunogen comprises a peptide sequence that is heterologous (i.e., non-native) to the VLP; that is, it is heterologous to the virus from which the components (including structural components) of the VLP are derived. As noted elsewhere herein, the VLP may be use as delivery vehicles for immunogens capable of raising an immune response to, for example, infectious agents or cancer proteins. It is to be understood that the choice of heterologous immunogen may depend on the therapeutic target and, hence, the condition to be treated or prevented. In an embodiment, the immunogen is selected from the group consisting of a virus antigen, or an immunogenic fragment or mimotope thereof, and a cancer-associated antigen, or an immunogenic fragment or mimotope thereof. In an embodiment, the immunogen is a heterologous virus antigen, or an immunogenic fragment or mimotope thereof.

In an embodiment, the immunogen is a cancer-associated antigen, or an immunogenic fragment or mimotope thereof. Suitable cancer-associated antigens will be familiar to persons skilled in the art, illustrative examples of which include antigens that are differentially expressed in bladder cancer, head and neck cancer, prostate cancer, breast cancer, lung cancer, ovarian cancer, pancreatic cancer, cancer of intestine and colon, stomach cancer, skin cancer and brain cancer; malignant diseases (Hodgkin's lymphoma and non-Hodgkin's lymphoma, etc.) that affect bone marrow (including leukemia) and lymphoproliferative system.

Other illustrative examples of cancer-associated antigens include MAGE (Science, 254: p 1643 (1991)), gp 100 (J. Exp. Med., 179: p 1005 (1994)), MART-1 (Proc. Natl. Acad. Sci. USA, 91: p 3515 (1994)), tyrosinase (J. Exp. Med., 178: p 489 (1993)), MAGE related proteins (J. Exp. Med., 179: p 921 (1994)), beta-catenin (J. Exp. Med, 183: p 1185 (1996)), CDK4 (Science, 269: p 1281 (1995)), HER2/neu (J. Exp. Med., 181: p 2109 (1995)), mutant p 53 (Proc. Natl. Acad. Sci. USA, 93: p 14704 (1996)), CEA (J. Natl. Cancer. Inst., 87: p 982 (1995)), PSA (J. Natl. Cancer. Inst., 89: p 293 (1997)), WT1 (Proc. Natl. Acad. Sci. USA, 101: p 13885 (2004)), HPV-derived antigen (J. Immunol., 154: p 5934 (1995)), EBV-derived antigen (Int. Immunol., 7: p 653 (1995)), MAGEA3 peptide 168-176 (Immunol. Rev. 188: 33 (2002)), gp 100 peptide 209-217 (Nat. Med. 4: 321 (1998)), gp 100 peptide 280-288 (Proc. Natl. Acad. Sci. USA 100: 8372 (2003)), Melan-A peptide 27-35 (Cancer J. Sci. Am. 3: 37 (1997)), Melan-A peptide 26-35, Tyrosinase peptide 1-9, Tyrosinase peptide 368-376, gp 100 peptide 280-288, gp 100 peptide 457-466 (Int. J. Cancer 67: 54 (1996)), HER-2 peptide 369-384, HER-2 peptide 688-703, HER-2 peptide 971-984 (J. Clin. Invest. 107: 477 (2001)), New York esophageal squamous cell carcinoma 1 (NY-ESO-1; see Front. Immunol.; 9: 947 (2018)) and MAGE-A12 peptide 170-178 (Int. J. Cancer 105: 210 (2003)).

In an embodiment, the immunogen comprises an antigen, or an antigenic fragment thereof, associated with a cancer selected from the group consisting of Adrenal Cancer, Anal Cancer, Bile Duct Cancer, Bladder Cancer, Bone Cancer, Brain/CNS Tumors in adults, Brain/CNS Tumors In Children, Breast Cancer, Breast Cancer In Men, Cancer in Adolescents, Cancer in Children, Cancer in Young Adults, Cancer of Unknown Primary, Castleman Disease, Cervical Cancer, Colon/Rectum Cancer, Endometrial Cancer, Esophagus Cancer, Ewing Family Of Tumors, Eye Cancer, Gallbladder Cancer, Gastrointestinal Carcinoid Tumors, Gastrointestinal Stromal Tumor, Gestational Trophoblastic Disease, Hodgkin Disease, Kaposi Sarcoma, Kidney Cancer, Laryngeal and Hypopharyngeal Cancer, Leukemia. Acute Lymphocytic in Adults, Leukemia, Acute Myeloid Leukemia, Chronic Lymphocytic Leukemia, Chronic Myeloid Leukemia, Chronic Myelomonocytic Leukemia, Leukemia in Children, Liver Cancer, Lung Cancer, Non-Small Cell Lung Cancer, Small Cell Lung Cancer, Lung Carcinoid Tumor, Lymphoma, Lymphoma of the Skin, Malignant Mesothelioma, Multiple Myeloma, Myelodysplastic Syndrome, Nasal Cavity and Paranasal Sinus Cancer, Nasopharyngeal Cancer, Neuroblastoma, Non-Hodgkin Lymphoma, Non-Hodgkin Lymphoma In Children, Oral Cavity and Oropharyngeal Cancer, Osteosarcoma, Ovarian Cancer, Pancreatic Cancer, Penile Cancer, Pituitary Tumors, Prostate Cancer, Retinoblastoma, Rhabdomyosarcoma, Salivary Gland Cancer, Adult Soft Tissue Cancer Sarcoma, Skin Cancer, Basal and Squamous Cell Skin Cancer, Melanoma Skin Cancer, Merkel Cell Skin cancer, Small Intestine Cancer, Stomach Cancer, Testicular Cancer, Thymus Cancer, Thyroid Cancer, Uterine Sarcoma, Vaginal Cancer, Vulvar Cancer, Waldenstrom Macroglobulinemia, and Wilms Tumor.

In an embodiment, the cancer is selected from the group consisting of breast cancer, gastric cancer, ovarian cancer, and uterine serous carcinoma.

It some embodiments, the immunogen is a non-structural (NS) viral protein. Suitable NS proteins will be familiar to those skilled in the art, illustrative examples of which are described elsewhere herein (e.g., HCV NS3).

In certain embodiments, it might be appropriate to employ two or more constructs. In some embodiments, each of the two or more construct comprises a nucleic acid sequence encoding a different component of the VLP that, collectively, are capable of self-assembling to form a VLP. For example, a vaccine composition may comprise a first construct comprising a nucleic acid sequence encoding the immunogen, as described herein, and a second construct comprising a nucleic acid sequence encoding a polyprotein comprising two or more viral structural proteins capable of forming a virus-like particle (VLP), wherein at least two of the two or more viral structural proteins are separated by a signal peptidase sequence, as described herein. The present inventor has surprisingly found that, by employing two or more constructs, as described herein, there is an unexpected improvement in the yield of VLP production. Thus, in an aspect disclosed herein, there is provided a system or a composition comprising (i) a first construct comprising a nucleic acid sequence encoding an immunogen, as described herein, and (ii) a second construct comprising a nucleic acid sequence encoding a polyprotein, wherein the polyprotein comprises two or more viral structural proteins capable of forming a virus-like particle (VLP), wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the immunogen and the liberated structural proteins to self-assemble into a VLP.

The term “system”, as used herein, typically refers to the first and second constructs as separate entities, permitting the first and second constructs to be introduced separately into a host cell. This advantageously allows the user to substitute, for example, the first construct with another first construct encoding a different immunogen whilst deploying the same second construct encoding the self-cleaving polyprotein of viral structural proteins for self-assembly with the immunogen to form the VLP.

In an embodiment, the system or composition may suitably comprise (i) a first construct comprising a nucleic acid sequence encoding a polyprotein comprising an immunogen, as described herein, and at least one viral structural protein capable of forming a virus-like particle (VLP), and (ii) a second construct comprising a nucleic acid sequence encoding at least one other viral structural protein capable of forming a VLP, wherein the immunogen and the at least one viral structural protein of the first construct are separated by a signal peptidase sequence, such that, when the polyprotein and the at least one other viral structural protein are expressed in a host cell, the signal peptidase sequence undergoes host cell peptidase-dependent cleavage to liberate the immunogen and the at least one viral structural protein, thereby allowing the liberated proteins to self-assemble with the at least one other viral structural protein so as to form a VLP.

In an embodiment, (i) the first construct comprises a nucleic acid sequence encoding a polyprotein comprising an immunogen and one viral structural protein, wherein the immunogen and the viral structural protein are separated by a signal peptidase sequence, such that, when the polyprotein is expressed in a host cell, the signal peptidase sequence undergoes host cell peptidase-dependent cleavage to liberate the immunogen and the viral structural protein, and (ii) the second construct comprises a nucleic acid sequence encoding at least one other viral structural protein, wherein the immunogen and viral structural proteins encoded by the first and second constructs, once liberated, including by host cell peptidase-dependent cleavage of the signal peptidase sequence(s), are capable of self-assembling to form a VLP.

In another embodiment, (i) the first construct comprises a nucleic acid sequence encoding a polyprotein comprising an immunogen and two or more viral structural proteins, wherein at least two of the immunogen and the two or more viral structural protein are separated by a signal peptidase sequence, such that, when the polyprotein is expressed in a host cell, the signal peptidase sequence undergoes host cell peptidase-dependent cleavage to liberate the immunogen and two or more viral structural proteins, and (ii) the second construct comprises a nucleic acid sequence encoding at least one other viral structural protein, wherein the immunogen and viral structural proteins encoded by the first and second constructs, once liberated, including by host cell peptidase-dependent cleavage of the signal peptidase sequence(s), are capable of self-assembling to form a VLP.

In an embodiment, the virus is of the family Flaviviridae, Coronaviridae, Orthomyxoviridae and Togaviridae. In an embodiment, the virus is of the family Flaviviridae.

In an embodiment, the virus is selected from the group consisting of a Flavivirus, a Hepacivirus, a Pegivirus and a Pestivirus. In an embodiment, the virus is a Flavivirus. In an embodiment, the Flavivirus is selected from the group consisting of a Zika virus and a Dengue virus.

In an embodiment, the virus is a Hepacivirus. In an embodiment, the virus is a Hepatitis C virus (HCV). In an embodiment, the two or more viral structural proteins are selected from the group consisting of an HCV core protein, a HCV envelope glycoprotein E1 and a HCV envelope glycoprotein E2. In an embodiment, the immunogen comprise an HCV NS3 protein. In an embodiment, the polyprotein comprises an HCV core protein, an HCV envelope glycoprotein E1, an HCV envelope glycoprotein E2 and an HCV NS3 protein.

In an embodiment, the Flavivirus is a Dengue virus. In an embodiment, the two or more viral structural proteins are selected from the group consisting of a Dengue core (capsid) protein, a Dengue membrane (prM) protein and a Dengue envelope (E) protein. In an embodiment, the polyprotein comprises a Dengue core (capsid) protein, a Dengue membrane (prM) protein and a Dengue envelope (E) protein.

In an embodiment, the virus is of the family Orthomyxoviridae. In an embodiment, the virus is an influenza virus. In an embodiment, the influenza virus is selected from the group consisting of influenza A, influenza B, influenza C and influenza D.

In an embodiment, the virus is of the family Togaviridae. In an embodiment, the virus is an Alphavirus.

In an embodiment, the immunogen is a heterologous immunogen. Illustrative examples of suitable heterologous immunogens are described elsewhere herein. In an embodiment, the immunogen is a viral antigen. Illustrative examples of suitable viral antigens are described elsewhere herein. In an embodiment, the immunogen is a non-viral antigen. Illustrative examples of suitable non-viral antigens are described elsewhere herein. In an embodiment, the heterologous non-viral antigen is a cancer-associated antigen. Illustrative examples of suitable cancer-associated antigens are described elsewhere herein.

In another aspect disclosed herein, there is provided a method of producing a VLP, the method comprising:
(i) introducing the first and second vaccine constructs, as described herein, into a host cell; and
(ii) culturing the host cell of (i) under conditions and for a period of time sufficient for the host cell to produce the VLP.

The present disclosure also extends to a VLP produced by the methods described herein, host cells and vaccine compositions comprising the vaccine constructs or the VLP described herein.

Suitable methods for preparing a nucleic acid sequence encoding the polyproteins disclosed herein will be familiar to persons skilled in the art, based on knowledge of the genetic code, possibly including optimising codons based on the nature of the host cell (e.g. microorganism) to be used for expressing and/or secreting the recombinant immunogen. Suitable host cells will also be known to persons skilled in the art, illustrative examples of which are described elsewhere herein and include prokaryotic cells (e.g., E. coli) and eukaryotic cells (e.g., P. pastoris). Reference is made to “Short Protocols in Molecular Biology, 5th Edition, 2 Volume Set: A Compendium of Methods from Current Protocols in Molecular Biology” (by Frederick M. Ausubel (author, editor), Roger Brent (editor), Robert E. Kingston (editor), David D. Moore (editor), J. G. Seidman (editor), John A. Smith (editor), Kevin Struhl (editor), J Wiley & Sons, London).

As used herein, the terms “encode,” “encoding” and the like refer to the capacity of a nucleic acid to provide for another nucleic acid or a polypeptide or polyprotein. For example, a nucleic acid sequence is said to “encode” a polypeptide or polyprotein if it can be transcribed and/or translated, typically in a host cell, to produce the polypeptide or polyprotein or if it can be processed into a form that can be transcribed and/or translated to produce the polypeptide or polyprotein. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product. In some embodiments, the nucleic acid sequence encoding the immunogens, the viral structural proteins and/or the signal peptidase sequences, as herein described, are codon-optimized for expression in a suitable host cell. For example, where the VLP are to be used for inducing an immune response in a human subject, the nucleic acid sequences encoding the immunogens, the viral structural proteins and/or the signal peptidase sequences, as herein described, can be human codon-optimized. Suitable methods for codon optimization would be known to persons skilled in the art, such as using the “Reverse Translation” option of “Gene Design” tool located in “Software Tools” on the John Hopkins University Build a Genome website.

### Host Cells

Suitable host cells permissive for viruses will be familiar to persons skilled in the art, illustrative examples of which are described in Sarkar et al. (Korean J. Microbiol. 2019; 55 (4):327-343), Santi et al. (Methods. 2006; 40(1): 66-76), Roldão et al. (Expert Rev. Vaccines; 2010; 9 (10): 1149-1176) and (Ghislain Masavuli et al. Front Microbiol. 2017; 8: 2413), and include bacteria, yeast, fungi, plant, insect, mammalian and avian cells.

In an embodiment, the host cell is a bacterium. Suitable virus permissive bacteria will be familiar to persons skilled in the art, an illustrative example of which includes Escherichia coli (Huang et al., NPJ Vaccines. 2017; 2:3).

In an embodiment, the host cell is a yeast cell. Suitable virus permissive yeast cells will be familiar to persons skilled in the art, illustrative examples of which are described in Kumar and Kumar (FEMS Yeast Research, 2019; 19(2): foz007) and include Pichia Pastoris (Kim and Kim, Letters Appl. Micro., 2017; 64(2):111-123). Saccharomyces cerevisiae (Zhao et al. Appl Microbiol Biotechnol. 2013; 97(24):10445-52) and Hansenula polymorpha (Wetzel et al. J Biotechnol. 2019; 20; 306:203-212). In an embodiment, the host cell is Pichia Pastoris. In an embodiment, the host cell is Saccharomyces cerevisiae. In an embodiment, the host cell is Hansenula polymorpha.

In an embodiment, the host cell is a plant cell. Suitable virus permissive plant cells will be familiar to persons skilled in the art, illustrative examples of which are described in Makarkov et al. (npj Vaccines; 2019; 4(17)) and Santi et al. (Methods. 2006; 40(1): 66-76), and include Nicotiana tabacum and Arabidopsis thaliana (Greco et al. Vaccine. 2007; 28; 25(49):8228-40), Samsun N N and Solanum tuberosum cv. Solara. In an embodiment, the host cell is Trichoplusia ni (BTI-TN-5B1-4). In an embodiment, the host cell is Nicotiana tabacum cv. Samsun N N. In an embodiment, the host cell is Solanum tuberosum cv. Solara.

In an embodiment, the host cell is an insect cell. Suitable virus permissive insect cells will be familiar to persons skilled in the art, illustrative examples of which include Spodoptera frugiperda (sf9; Wagner et al. PLoS One. 2014; 9(4):e94401), Trichoplusia ni (BTI-TNSB1-4; Krammer et al., Mol Biotechnol. 2010; 45(3):226-234) and Drosophila Schneider 2 (S2; Park et al., J Virol Methods. 2018; 261:156-159). In an embodiment, the host cell is Spodoptera frugiperda. In an embodiment, the host cell is Trichoplusia ni. In an embodiment, the host cell is Drosophila Schneider 2.

In an embodiment, the VLP is produced in a mammalian cell. Mammalian cells are used in expression of various proteins because of their ability to carry out the post translational modifications (PTM). An additional advantage of this system is that the proteins are secreted in their native, mature form. Suitable mammalian host cells will be familiar to persons skilled in the art, illustrative examples of which include Chinese hamster ovary (CHO) cells (Michel et al., 1984, Proc. Natl. Acad. Sci. USA. 81, 7708-7712; Patzer et al. 1986, J. Virol. 58, 884-892); Purdy and Chang, 2005, Virology. 333, 239-250), Vero cells, Human Embryonic Kidney 293 cells (Buffin et al., Vaccine, 2019; 37(46): 6857-6867) and Huh7 human liver cells (Earnest-Silveira et al., J Gen Virol. 2016; 97(8):1865-1876). In an embodiment, the host cell is Chinese hamster ovary (CHO). In an embodiment, the host cell is Human Embryonic Kidney 293. In an embodiment, the host cell is a Huh7 cell.

### VLP Production

The present disclosure also extends to methods of manufacturing the VLP described herein. Thus, in an aspect disclosed herein, there is provided a method of producing a modified virus-like particle (VLP), the method comprising:
(i) introducing the nucleic acid construct as described herein into a host cell; and
(ii) culturing the host cell of (i) under conditions and for a period of time sufficient for the host cell to produce the VLP.

Suitable culture methods and conditions/times suitable for producing VLP will be familiar to persons skilled in the art, understanding that the method steps and conditions/times will likely vary depending, for example, on the type of host cell employed. Illustrative examples of suitable methods of production are set out elsewhere herein and also described in Cheng and Mukhopadhyay (Virology, 2011; 413(2):153-160), Charlton Hume et al. (ibid), Ghislain Masavuli et al. (ibid) and Vacher et al. (Mol. Pharmaceutics 2013; 10:1596-1609).

Recombinant structural virus proteins (e.g., capsid proteins) which may be used to prepare the VLP described herein can be readily prepared by standard genetic engineering techniques by the skilled person provided with the sequence of the wild-type protein. Methods of genetically engineering proteins are well known in the art, illustrative examples of which are described, for example, in Ausubel et al. (1994 and updates) Current Protocols in Molecular Biology, John Wiley & Sons, New York).

Isolation and cloning of the nucleic acid sequence encoding the wild-type virus structural protein(s) can be achieved using standard techniques (see, e.g., Ausubel et al., ibid.). For example, the nucleic acid sequence can be obtained directly from the virus by extracting RNA by standard techniques and then synthesizing cDNA from the RNA template (e.g., by RT-PCR).

The nucleic acid sequence encoding the virus structural protein(s) is then inserted directly or after one or more subcloning steps into a suitable expression vector. Persons skilled in the art will understand that the precise vector used is not critical. Illustrative examples of suitable vectors include plasmids, phagemids, cosmids, bacteriophage, baculoviruses, retroviruses or DNA viruses. The structural protein(s) can then be expressed and purified as described in more detail below.

Alternatively, the nucleic acid sequence encoding the structural protein(s) can be further engineered to introduce one or more mutations, such as those described above, by standard in vitro site-directed mutagenesis techniques known to persons skilled in the art. Mutations can be introduced by deletion, insertion, substitution, inversion, or a combination thereof, of one or more of the appropriate nucleotides making up the coding sequence. This can be achieved, for example, by PCR based techniques for which primers are designed that incorporate one or more nucleotide mismatches, insertions or deletions. The presence of the mutation can be verified by a number of standard techniques, for example by restriction analysis or by DNA sequencing.

Virus proteins may also be engineered to produce fusion proteins comprising one or more immunogens fused to virus coat protein. Methods for making fusion proteins are well known to those skilled in the art. DNA sequences encoding a fusion protein can be inserted into a suitable expression vector as noted above. Persons skilled in the art will appreciate that the DNA encoding the coat protein or fusion protein can be altered in various ways without affecting the activity of the encoded protein. For example, variations in DNA sequence may be used to optimize for codon preference in a host cell used to express the protein, or may contain other sequence changes that facilitate expression.

It will be understood that the expression vector may further include regulatory elements, such as transcriptional elements, required for efficient transcription of the DNA sequence encoding the coat or fusion protein. Illustrative examples of suitable regulatory elements that can be incorporated into the vector include promoters, enhancers, terminators, and polyadenylation signals. The present disclosure therefore also provides vectors comprising a regulatory element operatively linked to one or more nucleic acid sequences encoding the VLP described herein. Persons skilled in the art will appreciate that selection of suitable regulatory elements may be dependent on the host cell chosen for expression of the VLP and that such regulatory elements may be derived from a variety of sources, including bacterial, fungal, viral, plant, mammalian or insect genes.

The expression vector may additionally contain heterologous nucleic acid sequences that facilitate the purification of the expressed VLP. Illustrative examples of suitable heterologous nucleic acid sequences include affinity tags such as metal-affinity tags, histidine tags, avidin/streptavidin encoding sequences, glutathione-S-transferase (GST) encoding sequences and biotin encoding sequences. The amino acids corresponding to expression of the nucleic acids can be removed from the expressed VLP prior to use, including clinical use, as described elsewhere herein. Alternatively, the amino acids corresponding to expression of heterologous nucleic acid sequences can be retained on the VLP if they do not interfere with subsequent use.

The expression vector can be introduced into a suitable host cell by one of a variety of methods known in the art, illustrative examples of which are generally described in Ausubel et al. (ibid.) and include, for example, stable or transient transfection, lipofection, electroporation, and infection with recombinant viral vectors. One skilled in the art will understand that selection of the appropriate host cell for expression of the coat protein will be dependent upon the vector chosen. Illustrative examples of suitable host cells are described elsewhere herein and include bacterial, yeast, insect, plant and mammalian cells.

It is to be understood that the recombinant viral structural proteins should be capable of multimerization and assembly into VLP. In general, assembly takes place in the host cell expressing the structural proteins. The VLP can be isolated from the host cells by standard techniques known to persons skilled in the art, such as those described elsewhere herein. The VLP can be further purified by standard techniques, such as chromatography, to remove contaminating host cell proteins or other compounds.

Methods for introducing the nucleic acid construct into a host cell for the purposes of producing VLP will be known to persons skilled in the art, illustrative examples of which include transformation, transduction, electroporation, conjugation, transfection, calcium phosphate methods, and the like.

Host cells expressing one or more of the sequences described herein can readily be generated given the disclosure provided herein by stably integrating the sequences into the genome of a host cell, to allow for the formation of the VLP, as described herein. The promoter regulating expression of the stably integrated nucleic acid sequences(s) may be constitutive or inducible. Thus, a host cell can be generated in which the VLP proteins are stably integrated such that, upon introduction of the nucleic acid-encoding sequences or expression construct comprising these virus sequences into the host cell of the proteins encoded by said nucleic acid sequences, form non-replicating VLP.

When the genes that code for the proteins required for VLP formation are introduced into a host cell and subsequently expressed at the necessary level, the VLP assembles and can then be released from the cell into the culture media, where it can be further processed (e.g., purified), if necessary, having regard to the intended use.

Depending on the expression system and host cell selected, the VLP, as herein defined, are typically produced by growing the host cell transformed by the expression vector under conditions whereby the viral proteins encoded by the construct/expression vector are expressed and VLP can be formed. The selection of the appropriate growth conditions will be familiar to persons skilled in the art.

The VLP can be isolated (or substantially purified) using methods that preserve the integrity thereof, such as, by density gradient centrifugation, e.g., sucrose gradients, PEG-precipitation, pelleting, and the like (see, e.g., Kimbauer et al. J. Virol. (1993) 67:6929-6936), as well as standard purification techniques including, e.g., ion exchange and gel filtration chromatography. For example, a composition or preparation comprising an isolated VLP prepared according to the method of the present disclosure may comprise at least 65%, at least 70%, at least 75%, at least 80%, at least 85%, at least 90%, at least 95%, at least 96%, at least 98%, at least 99% or 100% of an isolated VLP, as measured by methods known to persons skilled in the art.

The presence of the VLP can be detected using conventional techniques known in the art, such as by electron microscopy, atomic force microscopy, biophysical characterization, and the like (see, e.g., Baker et al., Biophys. J. (1991) 60:1445-1456; and Hagensee et al., J. Virol. (1994) 68:4503-4505). For example, the VLP can be isolated by density gradient centrifugation and/or identified by characteristic density banding. Alternatively, cryoelectron microscopy can be performed on vitrified aqueous samples of the VLP preparation in question, and images recorded under appropriate exposure conditions.

The present disclosure also extends to a VLP produced by the methods described herein and a vaccine composition comprising the vaccine constructs and/or VLP, as described herein.

### Methods of Treatment

The vaccine constructs, VLP and compositions described herein may be used to potentiate an immune response in an animal, or as vaccines to induce a protective or therapeutic immune response to an immunogen in a host. Thus, in another aspect disclosed herein, there is provided a method of raising an immune response in a subject to an immunogen, the method comprising administering to a subject in need thereof the vaccine construct, VLP or composition described herein. In another aspect disclosed herein, there is provided use of the vaccine construct, VLP or composition described herein in the manufacture of a medicament for raising an immune response in a subject to the immunogen. In an embodiment, the immune response generates antibodies to the immunogen. In yet another aspect disclosed herein, there is provided the vaccine construct, VLP or composition described herein for use in raising an immune response in a subject against the immunogen. In an embodiment of the present invention, the vaccine construct, VLP or composition described herein induce a humoral, cellular and innate immune response in the subject to which they are administered.

The vaccine constructs or VLP described herein may be advantageously formulated as pharmaceutical compositions together with a pharmaceutically acceptable carrier, diluent, and/or excipient. Suitable carriers, diluents, and/or excipients are well known in the art. If desired, an adjuvant or other active ingredient optionally may be included in the compositions. In an embodiment, the compositions are capable of efficiently potentiating an immune response in the absence of an additional adjuvant.

For administration to a host, the compositions described herein can be formulated for administration by a variety of routes. For example, the compositions can be formulated for oral, topical, rectal or parenteral administration or for administration by inhalation, intranasally or spray. The term “parenteral”, as used herein, includes subcutaneous injections, intradermal, intravenous, intramuscular, intrathecal, intrasternal injection and infusion techniques. In an embodiment, the compositions are formulated for topical, rectal or parenteral administration or for administration by inhalation, intranasally or spray. In an embodiment, the compositions are formulated for parenteral administration.

The compositions described herein will suitably comprise a therapeutically effective amount of the vaccine construct or VLP. The phrase “therapeutically effective amount” typically means an amount of the vaccine construct and/or VLP, as described herein, necessary to attain the desired response, for example, the inducement of an immune response to the target antigen. Typically, the appropriate dosage of the vaccine construct and/or VLP, as described herein, may depend on a variety of factors including, but not limited to, a subject's physical characteristics (e.g., age, weight, sex), whether the vaccine construct and/or VLP, as described herein, is being used as single agent or as part of adjuvant therapy, the progression (i.e., pathological state) of any underlying virus infection or disease, and other factors that may be recognized by persons skilled in the art. Various general considerations that may be considered when determining, for example, an appropriate dosage of the vaccine composition (see, e.g., in Gennaro (2000) “Remington: The Science and Practice of Pharmacy”, 20th edition, Lippincott, Williams, & Wilkins; and Gilman et al., (Eds), (1990), “Goodman And Gilman's: The Pharmacological Bases of Therapeutics”, Pergamon Press). It is expected that the amount will fall in a relatively broad range that can be determined through methods known to persons skilled in the art. Illustrative examples of a suitable therapeutically effective amount of VLP for administration to a human subject include from about 0.001 mg per kg of body weight to about 1 g per kg of body weight, preferably from about 0.001 mg per kg of body weight to about 50 g per kg of body weight, more preferably from about 0.01 mg per kg of body weight to about 1.0 mg per kg of body weight. Dosage regimes may be adjusted to provide the optimum therapeutic response. For example, several divided doses may be administered daily, weekly, monthly or other suitable time intervals, or the dose may be proportionally reduced as indicated by the exigencies of the situation.

In an embodiment, a therapeutically effective amount of the vaccine construct and/or VLP, as described herein, as defined herein, is effective to induce an immune response to the target antigen irrespective of genotype.

As described elsewhere herein, the terms “immune response””, “immunological response” and the like are typically used herein to refer to the development in a subject of a humoral and/or a cellular immune response to the target antigen. A “humoral immune response” typically refers to an immune response mediated by antibody molecules, while a “cellular immune response” is typically mediated by T-lymphocytes and/or other white blood cells. In a non-limiting example, the vaccine construct and/or VLP, as described herein, when administered to a subject induces an immune response selected from one or more of a neutralising antibody response, a cytotoxic T lymphocyte (CTL) response, a natural killer T cell response and/or a helper T lymphocyte (e.g., CD4+ T cell) response and innate immune response to the target antigen.

Methods for measuring an immune response will be known to persons skilled in the art, illustrative examples of which include plaque-reduction neutralization assay, micro-neutralization assay, solid-phase heterogeneous assays (e.g., enzyme-linked immunosorbent assay), solution phase assays (e.g., electrochemiluminescence assay), Western immunoblot, amplified luminescent proximity homogeneous assays, flow cytometry, intracellular cytokine staining, functional T-cell assays including suppressor T-cell assays, functional B-cell assays, functional monocyte-macrophage assays, dendritic and reticular endothelial cell assays, measurement of NK or NKT cell responses, oxidative burst assays, cytotoxic-specific cell lysis assays, pentamer binding assays, and phagocytosis and apoptosis evaluation.

As described elsewhere herein, the vaccine construct and/or VLP, as described herein, can be administered to a subject in need thereof by any suitable route of administration, including administration of the composition orally, nasally, nasopharyngeally, parenterally, enterically, gastrically, topically, transdermally, subcutaneously, intramuscularly, intradermally, in tablet, solid, powdered, liquid, aerosol form, locally or systemically, with or without added excipients. Suitable methods for preparing parenterally administrable compositions will be known or apparent to those skilled in the art and are described in more detail in such publications as Remington's Pharmaceutical Science, 15th ed., Mack Publishing Company, Easton, Pa. (1980). In some instances, administration is accomplished by intramuscular injection of the vaccine construct and/or VLP. In general, the vaccine construct and/or VLP, as described herein, can be administered in a manner compatible with the route of administration and physical characteristics of the recipient (including health status) and in such a way that it elicits the desired effect(s) (e.g. the induction of a protective immune response against the target antigen).

The vaccine construct and/or VLP, as described herein, may be administered to a recipient in isolation or in combination with other additional therapeutic agent(s). In embodiments where a pharmaceutical composition comprising the vaccine construct and/or VLP, as described herein, is formulated for administration with additional therapeutic agent(s), the administration may be simultaneous or sequential (i.e., administration of the vaccine construct and/or VLP is followed by administration of the additional agent(s) or vice versa). Thus, here two or more entities are administered to a subject “in conjunction”, they may be administered in a single composition at the same time, or in separate compositions at the same time, or in separate compositions separated in time.

In a non-limiting example, the vaccine construct and/or VLP, as described herein, may be administered in conjunction with an antiviral agent. An “antiviral agent or compound” is defined as an agent which, when administered to a subject, is capable of significantly reducing the virus titer in the blood or serum either directly (e.g., by inhibiting a viral enzyme activity) or indirectly (e.g., via modulation of the antiviral responses of a host cell), either transiently or in a sustained way. In general, the term antiviral agents comprise any pharmaceutically acceptable form of said agents including pharmaceutically acceptable salts and solvents as long as the biological effectiveness of the antiviral agent is not significantly compromised. Suitable antiviral agents would be known to persons skilled in the art, illustrative examples of which include sofosbuvir, ledipasvir, ribavirin, paritaprevir, and simeprevir.

In an embodiment, the immune response that is raised by the methods, uses and compositions described herein comprises an innate immune response. In an embodiment, the innate immune response comprises activation of NKT cells. In an embodiment, the immune response further comprises an adaptive immune response. In an embodiment, the adaptive immune response comprises activation of adaptive CD4 and/or CD8 T lymphocytes.

Various pharmaceutical compositions suitable for different routes of administration and methods of preparing pharmaceutical compositions will be known in the art, illustrative examples of which are described in “Remington: The Science and Practice of Pharmacy” (formerly “Remingtons Pharmaceutical Sciences”); Gennaro, A., Lippincott, Williams & Wilkins, Philadelphia, Pa. (2000).

In an embodiment, vaccine construct and/or VLP, as described herein, are suitable for the treatment or prevention of a cancer, a cytopathic viral infection (such as infection by a coronavirus, hepatitis B virus (HBV), hepatitis C virus (HCV) or human immunodeficiency virus (HIV)-1), a viral disease or a intracellular pathogen infection in a subject.

In an embodiment, the vaccine construct and/or VLP, as described herein, may suitably be given in an appropriate single dosage in order to elicit an immune response. In other embodiments, the initial dose may be followed by boosting dose. The boosting dose may comprise the same vaccine construct and/or VLP as the initial (priming) dose, whether at an equivalent dose (e.g., the same or similar dose), a lower dose or a higher dose as compared to the initial dose, or it may comprise the immunogen alone, or the immunogen in combination with a suitable adjuvant, as described elsewhere herein; that is, in the absence of the vaccine construct and/or VLP.

The administration regime need not differ from any other generally accepted vaccination programs. For instance, a single administration in an amount sufficient to elicit an effective immune response may be used. Alternatively, as noted above, other regimes of initial administration of the complex followed by boosting with immunogen alone, including as described above. Boosting may occur at times that take place well after the initial administration if the immune response (as measured, e.g., by antibody titres) falls below acceptable levels.

Alternatively, or in addition, the vaccine construct and/or VLP, as described herein, can be used in combination an additional immunopotentiator or adjuvant to enhance an immune response in humans or non-human animals against the targeted antigen(s). The present disclosure therefore extends to compositions further comprising an immunopotentiator or adjuvant. Preferably, the immunopotentiator or adjuvant is administered concomitantly with the vaccine construct and/or VLP, as described herein. The immunopotentiator or adjuvant can be administered prior or subsequently to the vaccine construct and/or VLP, as described herein, depending on the need as can be suitably determined by persons skilled in the art. The term “immunopotentiator,” as used herein, is intended to mean a substance that, when mixed with an immunogen, elicits a greater immune response than the immunogen alone. For example, an immunopotentiator can enhance immunogenicity and provide a superior immune response. An immunopotentiator can act, for example, by enhancing the expression of co-stimulators on macrophages and other antigen-presenting cells.

Suitable immunopotentiators or adjuvants will be familiar to persons skilled in the art, illustrative examples of which include 1018 ISS, aluminum salts, AMPLIVAX.RTM., AS15, BCG, CP-870,893, CpG7909, CyaA, dSLIM, flagellin or TLRS ligands derived from flagellin, FLT3 ligand, GM-CSF, IC30, IC31, Imiquimod (ALDARA.RTM.), resiquimod, ImuFact IMP321, Interleukins such as IL-2, IL-12, IL-18, IL-21, Interferon-alpha or -beta or -gamma or pegylated derivatives thereof, IS Patch, ISS, ISCOMATRIX, ISCOMs, JuvImmune.RTM., LipoVac, MALP2, MF59, monophosphoryl lipid A, Montanide IMS 1312, Montanide ISA 206, Montanide ISA 50V, Montanide ISA-51, other suitable water-in-oil and oil-in-water emulsions (e.g., AddaVax), OK-432, OM-174, OM-197-MP-EC, ONTAK, OspA, PepTel.RTM. vector system, poly(lactid co-glycolid) [PLG]-based and dextran microparticles, talactoferrin SRL172, YF-17D, VEGF trap, R848, beta-glucan, Pam3Cys, Aquila's QS21 stimulon, which is derived from saponin, mycobacterial extracts and synthetic bacterial cell wall mimics, and other proprietary adjuvants such as Ribi's Detox, Quil, or Superfos. Adjuvants such as Freund's or GM-CSF are preferred. Several immunological adjuvants (e.g., MF59) specific for dendritic cells and their preparation have been described previously (Allison and Krummel, 1995). Immunopotentiating cytokines may also be used. Several cytokines have been directly linked to influencing dendritic cell migration to lymphoid tissues (e.g., TNF-), accelerating the maturation of dendritic cells into efficient antigen-presenting cells for T-lymphocytes (e.g., GM-CSF, IL-1 and IL-4) (see, e.g., U.S. Pat. No. 5,849,589, specifically incorporated herein by reference in its entirety) and acting as immunoadjuvants (e.g., IL-12, IL-15, IL-23, IL-7, IL-18, IFN-alpha. IFN-beta).

CpG immunostimulatory oligonucleotides have also been reported to enhance the effects of adjuvants in a vaccine setting. Without being bound by theory or by a mode of application, CpG oligonucleotides act by activating the innate (non-adaptive) immune system via Toll-like receptors (TLR), mainly TLR9. CpG triggered TLR9 activation enhances antigen-specific humoral and cellular responses to a wide variety of antigens, including peptide or protein antigens, live or killed viruses, dendritic cell vaccines, autologous cellular vaccines and polysaccharide conjugates in both prophylactic and therapeutic vaccines. More importantly, it can enhance dendritic cell maturation and differentiation, resulting in enhanced activation of TH1 cells and strong cytotoxic T-lymphocyte (CTL) generation, even in the absence of CD4 T cell help. The TH1 bias induced by TLR9 stimulation is maintained even in the presence of vaccine adjuvants such as alum or incomplete Freund's adjuvant (IFA) that normally promote a TH2 bias. CpG oligonucleotides show even greater adjuvant activity when formulated or co-administered with other adjuvants or in formulations such as microparticles, nanoparticles, lipid emulsions or similar formulations, which are especially necessary for inducing a strong response when the antigen is relatively weak. They also accelerate the immune response and enable the antigen doses to be reduced by approximately two orders of magnitude, with comparable antibody responses to the full-dose vaccine without CpG. U.S. Pat. No. 6,406,705 describes the combined use of CpG oligonucleotides, non-nucleic acid adjuvants and an antigen to induce an antigen-specific immune response. A CpG TLR9 antagonist is dSLIM (double Stem Loop Immunomodulator) by Mologen (Berlin, Germany) which is a preferred component of the pharmaceutical composition of the present invention. Other TLR binding molecules such as RNA binding TLR 7, TLR 8 and/or TLR 9 may also be used.

Other illustrative examples of suitable adjuvants include chemically modified CpGs (e.g. CpR, Idera), dsRNA analogues such as Poly(I:C) and derivates thereof (e.g. AmpliGen.RTM., Hiltonol.RTM., poly-(ICLC), poly(IC-R), poly(I:C12U), non-CpG bacterial DNA or RNA as well as immunoactive small molecules and antibodies such as cyclophosphamide, sunitinib, Bevacizumab.RTM., celebrex, NCX-4016, sildenafil, tadalafil, vardenafil, sorafenib, temozolomide, temsirolimus, XL-999, CP-547632, pazopanib, VEGF Trap, ZD2171, AZD2171, anti-CTLA4, other antibodies targeting key structures of the immune system (e.g. anti-CD40, anti-TGFbeta, anti-TNF receptor) and SC58175, which may act therapeutically and/or as an adjuvant. The amounts and concentrations of adjuvants and additives useful in the context of the present invention can readily be determined by the skilled artisan without undue experimentation.

In an embodiment, the adjuvant is selected from the group consisting of anti-CD40, imiquimod, resiquimod, GM-CSF, cyclophosphamide, sunitinib, bevacizumab, interferon-alpha, CpG oligonucleotides and derivates, poly-(I:C) and derivates, RNA, sildenafil, and particulate formulations with PLG. In an embodiment, the adjuvant is an oil-in-water emulsion or an immunostimulatory lipid.

In an embodiment, the compositions described herein further comprise an immunostimulatory or adjuvanting lipid. Suitable immunostimulatory lipids will be familiar to persons skilled in the art, illustrative examples of which include glycolipids and phospholipids. In an embodiment, the immunostimulatory lipid is an immunostimulatory glycolipid or an immunostimulatory phospholipid. The immunostimulatory lipid may be a naturally-occurring lipid or it may be synthetically derived (synthesized). In some embodiments, the immunostimulatory lipid is a synthetic lipid. In some embodiments, the synthetic lipid may resemble, in part or in whole, a naturally-occurring lipid. Thus, also contemplated herein are immunostimulatory lipid analogues; that is, synthetic immunostimulatory lipids that resemble, at least in part, naturally-occurring lipids and yet are still capable of acting as an adjuvant to potentiate a host's immune response to an immunogen. Suitable immunostimulatory lipid analogues will be familiar to persons skilled in the art, illustrative examples of which are described in US patent publication nos. 20200009165 and 20190358318, Maeda et al. (Vaccine; 1989; 7(3):275-281); Jiang et al. (Carbohydr. Res., 2007; 342(6):784-796); Foster et al. (J. Med. Chem., 2018; 61(3):1045-1060), the contents of which are incorporated herein by reference in their entirety.

In an embodiment, the immunostimulatory lipid is a glycolipid. Suitable immunostimulatory glycolipids will be familiar to persons skilled in the art, illustrative examples of which are described in Kim et al. (Expert Rev Vaccines. 2008; 7(10): 1519-1532), Godfrey, et al. (Nat. Immunol 2015; 16: 1114-1123, and Cerundolo, et al. (Nat. Rev. Immunol. 2009; 9:28-38). In an embodiment, the glycolipid is an alpha-anomeric glycolipid or a beta-anomeric glycolipid. In an embodiment, the glycolipid is an alpha-anomeric glycolipid. In an embodiment, the alpha-anomeric glycolipid is an alpha-anomeric glycosphingolipid. In an embodiment, the alpha-anomeric glycosphingolipid is α-galactosylceramide or α-glucosylceramide. In another embodiment, the alpha-anomeric glycosphingolipid is α-glucosylceramide. In another embodiment, the glycolipid is a beta-anomeric glycolipid. In another embodiment, the beta-anomeric glycolipid is a beta-anomeric glycosphingolipid. In another embodiment, the beta-anomeric glycolipid is selected from the group consisting of β-mannosylceramide, β-glucosylceramide and β-galactosylceramide. In another embodiment, the beta-anomeric glycolipid is β-mannosylceramide.

In another embodiment, the immunostimulatory lipid is a phospholipid. Suitable immunostimulatory phospholipids will be familiar to persons skilled in the art, illustrative examples of which are described in Godfrey, et al. (2015; ibid). In an embodiment, the phospholipid is lyso-phosphatidylcholine or lysophosphatidyl-ethanolamine. Immunostimulatory lipids, as described herein, including immunostimulatory glycolipids and phospholipids, can vary in the length and saturation of their fatty acid chains (including the acyl and/or sphingosine chains). These are typically referred to by reference to the number of carbons on the acyl chain (e.g., α-GalCer C26, α-GalCer C24, α-GalCer C20:2 etc). For instance, α-GalCer, also known as KRN7000, has an 18C phytosphingosine and a 26C acyl chain, whereas α-GalCer C20:2 has a C20 acyl chain and cis-diunsaturation at C11 and C14. Suitable immunostimulatory lipids of varying length and saturation of their fatty acid chains will be familiar to persons skilled in the art,

illustrative examples are described in Wun et al. (2011. Immunity 34: 327-339). Such variation in the length and saturation of their fatty acid chains may impact on the immunostimulatory capacity of such lipids. Nevertheless, it is to be understood that the immunostimulatory lipids described herein, when administered to a host with an immunogen, will be capable of potentiating the host's immune response to the immunogen, irrespective of the length and saturation of their fatty acid chains. Methods of determining whether an immunostimulatory lipid is capable of potentiating the host's immune response to an immunogen according to the methods described herein, irrespective of the length and saturation of their fatty acid chains, will be known to persons skilled in the art.

In another embodiment, the immunostimulatory lipid is a natural killer T (NKT) cell agonist. In an embodiment, the immunostimulatory lipid is a type 1 and a type 2 NKT cell agonist. In an embodiment, the immunostimulatory lipid is a type 1 NKT cell agonist. In an embodiment, the immunostimulatory lipid is a type 2 NKT cell agonist. Type 1 and type 2 NKT cells are known in the art (see, e.g., Godfrey et al., 2015, ibid). Illustrative examples of suitable lipids that are capable of activating NKT cells are described in Kim et al. (ibid) and Godfrey et al. (2015, ibid), including those described elsewhere herein.

The terms “immunisation” and “vaccination” are used interchangeably herein to refer to the administration of the vaccine construct and/or VLP, as described herein, to a subject for the purposes of raising an immune response and can have a prophylactic effect, a therapeutic effect, or a combination thereof.

As used herein, the terms “treat,” “treated,” or “treating” when used with respect to a disease or pathogen refers to a treatment which increases the resistance of a subject to the disease or to infection with a pathogen (i.e. decreases the likelihood that the subject will contract the disease or become infected with the pathogen) as well as a treatment after the subject has contracted the disease or become infected in order to fight a disease or infection (e.g., to reduce, eliminate, ameliorate or otherwise stabilise a disease or infection). In an embodiment, the vaccine construct and/or VLP, as described herein, are capable of providing protective immunity to a host. The term “protective immunity,” as used herein, is intended to mean the ability of a host, such as a mammal, bird, or fish, to resist (delayed onset of symptoms, reduced severity of symptoms or lack of symptoms), as a result of its exposure to the vaccine construct and/or VLP, as described herein, disease or death that would otherwise follow exposure to a pathogen. Protective immunity is typically achieved by one or more of mucosal, humoral, or cellular immunity. Mucosal immunity is understood to mean the presence of secretory IgA (sIgA) antibodies on mucosal surfaces of the respiratory, gastrointestinal, and genitourinary tracts. Humoral immunity typically refers to the presence of IgG antibodies and IgM antibodies in serum. Cellular immunity typically refers to activation of cytotoxic T lymphocytes or delayed-type hypersensitivity that involves macrophages and T lymphocytes, as well as other mechanisms involving T cells without a requirement for antibodies.

The term “subject”, “host” or “patient” refers to an animal in need of treatment. The subject, host or patient encompasses human and non-human subjects, including, but not limited to, mammals, birds and fish, and suitably encompasses domestic, farm, zoo and wild animals, such as, for example, cows, pigs, horses, goats, sheep or other hoofed animals, dogs, cats, chickens, ducks, non-human primates, guinea pigs, rabbits, ferrets, rats, hamsters and mice.

The present disclosure also extends to pharmaceutical kits or packs comprising the vaccine construct, VLP and/or compositions, as described herein, including for use as a vaccine. Individual components of the kit can be packaged in separate containers, associated with which, when applicable, can be a notice in the form prescribed by a governmental agency regulating the manufacture, use or sale of pharmaceuticals or biological products, which notice reflects approval by the agency of manufacture, use or sale for human or animal administration.

The kits may optionally further comprise one or more other therapeutic agents for use in combination with the vaccine construct, VLP and/or compositions, as described herein. The kit may optionally contain instructions or directions outlining the method of use or administration regimen.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also suitably be provided in dried or lyophilised form and the kit can additionally contain a suitable solvent for reconstitution of the lyophilised components. Irrespective of the number or type of containers, the kits of the invention also may comprise an instrument for assisting with the administration of the composition to a patient. Such an instrument may be an inhalant, syringe, pipette, forceps, measured spoon, eye dropper or similar medically approved delivery vehicle.

All patents, patent applications and publications mentioned herein are hereby incorporated by reference in their entireties.

Aspects disclosed herein are further described by the following non-limiting examples.

### EXAMPLES

### Example 1—Vaccine Construct Encoding a Self-Cleaving Multivalent HCV VLP Construct—AdenoHCV1aCE1NS3E2

As noted elsewhere herein, current approaches to VLP, in particular multivalent VLP carrying multiple heterologous antigens of different pathogens unrelated to the structural components of the VLP, are generally limited in how they will produce efficacious vaccines capable of (1) producing potent neutralising antibodies that will not wane quickly and possibly raise the risk of antibody-dependent enhancement (ADE) of infection; and (2) protecting against vaccine escape mutants that will arise through the selection of variants that are not efficiently neutralised by antibodies raised against the heterologous antigens of the VLP, both of which can result in neutralisation escape and a loss of vaccine efficacy. To address these issues, the inventor has generated a self-cleaving VLP platform that can be conveniently modified, as required, to target variant viruses as they emerge.

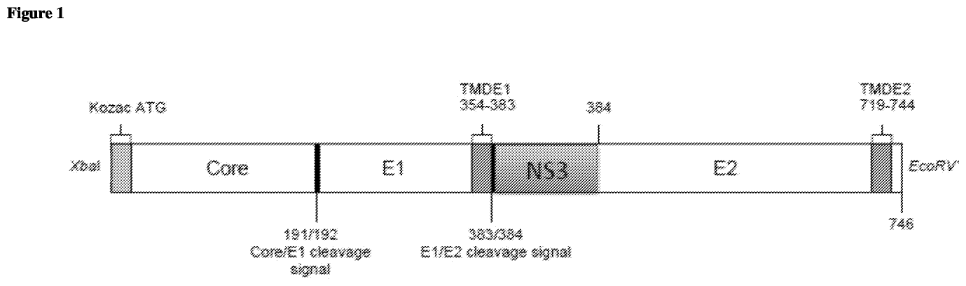

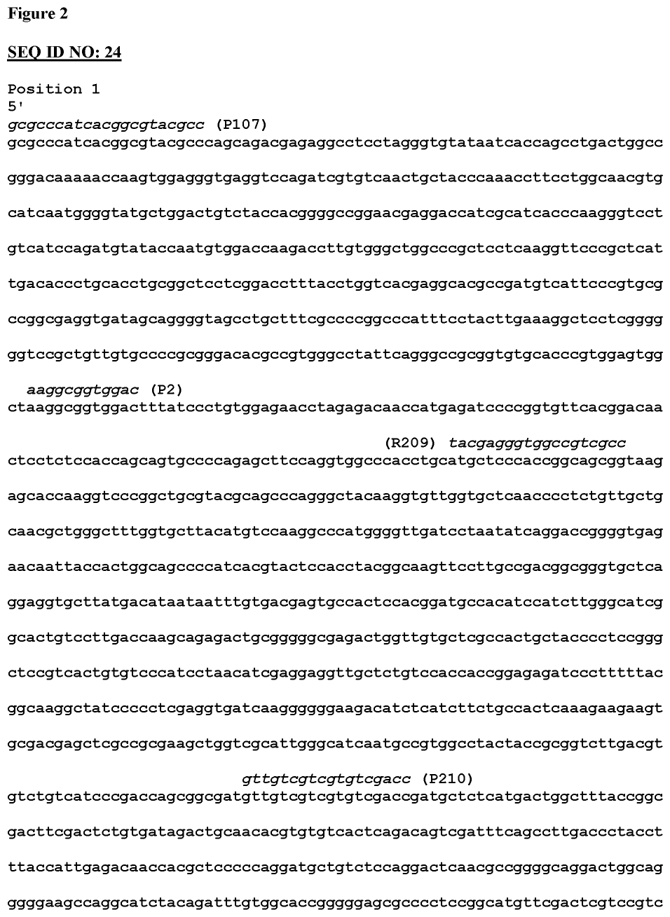

In this illustrative example, a vaccine construct was developed that is capable of producing a self-cleaving polyprotein comprising the HCV core (capsid), E1, E2 and NS3 structural proteins. A diagrammatical representation of the polyprotein encoded by this multivalent HCV vaccine construct is shown in FIG. 1.

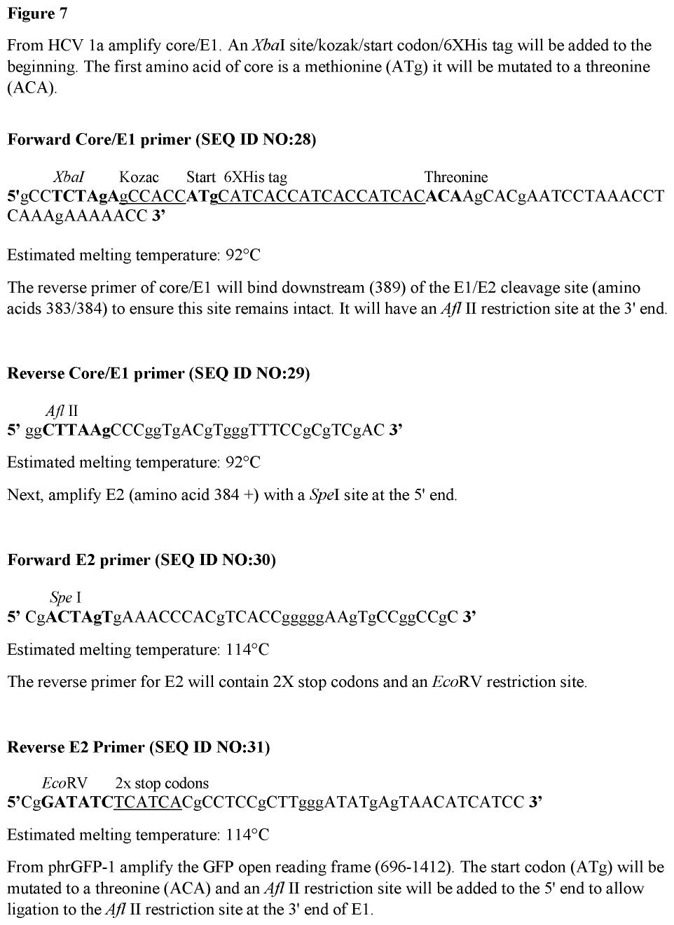

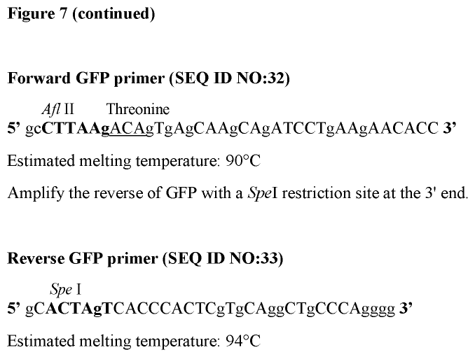

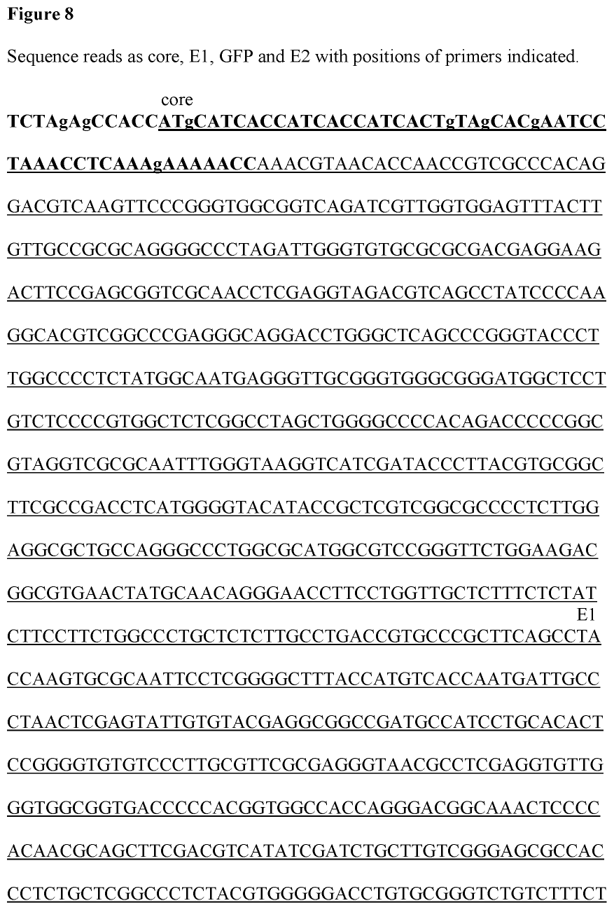

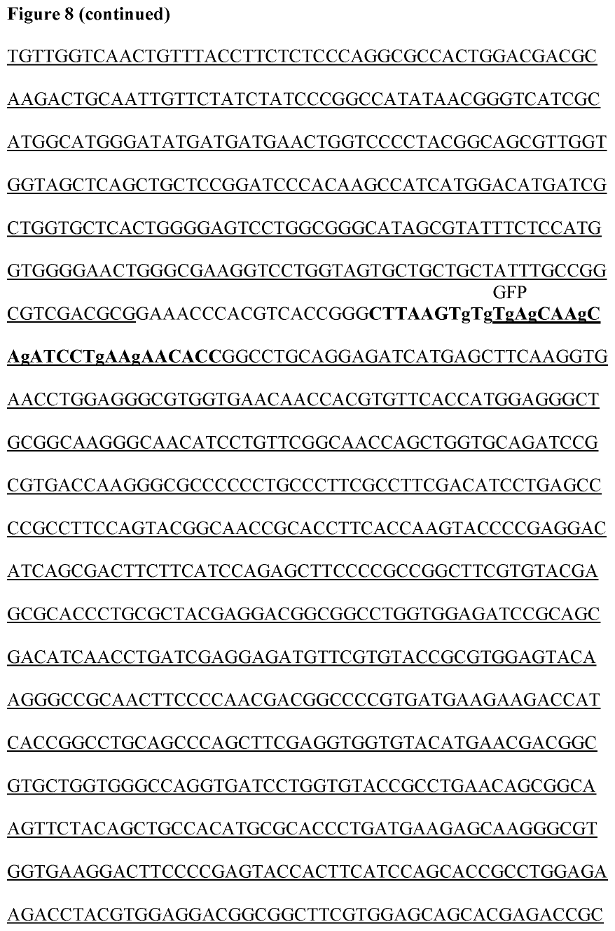

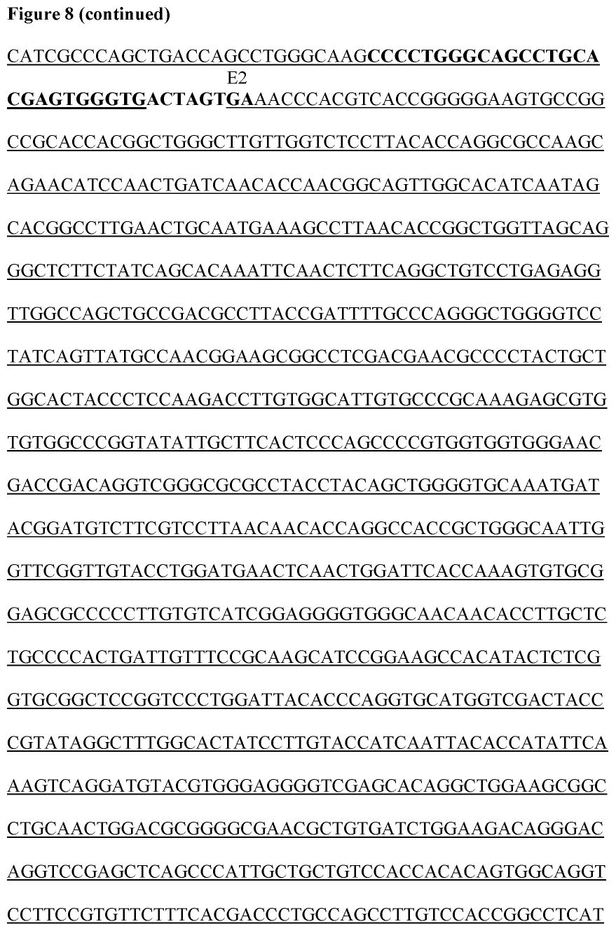

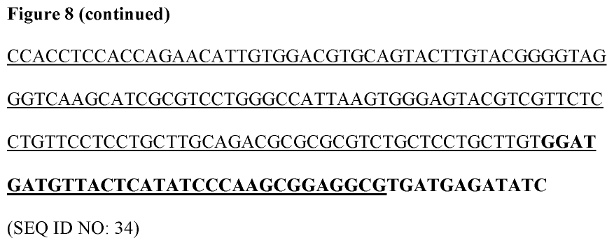

A restriction digestion of the pAdTHCV1aCE1GFPE2 construct was performed to release the GFP insert. The cloning strategy for the pAdTHCV1aCE1GFPE2 construct, and its sequence is provided in FIGS. 7 and 8, respectively.

About 5 μg of pAdTHCV1aCE1GFPE2 plasmid DNA (10 μl) was digested in the presence of AflII (1 μl), SpeI (1 μl), H₂O (6 μl) and 10 x restriction digest buffer (2 μl). The digests were then run on a 1% TAE agarose gel and the product bands visualised. Two bands were visualised:
1. pAdTHCV1aCE1E2 linearized vector (˜11.5 kb)
2. GFP insert (˜750 bp)

The 11.5 kb band was gel purified for ligation with NS3 using QIAquick Gel Extraction Kit according to the manufacturer's instructions.

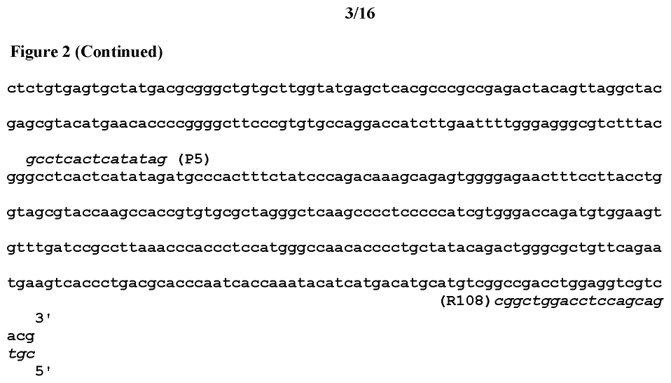

cDNA encoding the HCV NS3 structural protein was produced by PCR amplification from HCV H771a (GenBank Accession No. AF009606). The forward (F107) and reverse (R108) primers employed for amplification of NS3 cDNA are shown in FIG. 2.

Conditions employed for NS3 cDNA amplification are set out below:

### PCR Amplification Reagents

10 x buffer with 15 mMMgCl2: 5 μl
2.5 mM dNTP mix: 5 μl
Forward primer F107 (50 ng/μl): 5 μl
Reverse primer R108 (50 ng/μl): 5 μl
DNA: 1 μl
H₂O: 25 μl
HiFi Taq polymerase: 1 μl

### Cycling Conditions

1. 95° C.—5 mins
2. 95° C.—45 secs
3. 45° C.—45 secs
4. 72° C.—2 mins; 30 cycles of Step 2 to 4
5. 72° C.—10 mins for final extension
6. 40° C.—end

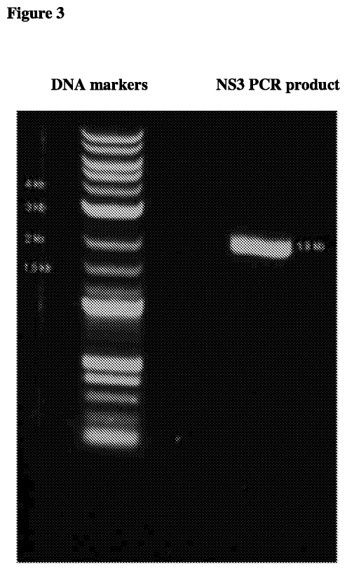

NS3 PCR product (at ˜1,893 kb) is shown in the photomicrograph of FIG. 3.

The NS3 amplification product was ligated into a pTOPO2.1 plasmid using pTOPO kit (TOPO™ TA Cloning™ Kit for Subcloning), with TOP10F′ E. coli, employing the following reagents and conditions:
PCR product NS3: 2 μl
Salt (1.2 M NaCl; 0.06 M MgCl₂): 1 μl
dH₂O: 2 μl
TOPO vector: 1 μl
Ligation at 16° C. for 2 hours.

The ligation product was then transform into chemically competent cells TOP10F′ cells. Briefly, 10 μl X-Gal Solution (20 mg/ml) and 10 μl IPTG (100 mM) were added per 1 mL of cell culture medium. The cells were then cultured in the medium overnight at 37° C. White cell colonies were selected and transferred to Luria Broth (LB)/Amp (50 μg/ml final) where they were grown overnight at 37° C. Plasmids were then extracted from the cultures using QIAprep Spin Miniprep Kit according to the manufacturer's instructions. To confirm the presence of ligated NS3 inserts in the pTOPO2.1 plasmid, restriction digests were performed with AFlII and SpeI using the following conditions and reagents:
Plasmid DNA: 10 μl
AflII: 1 μl
SpeI: 1 μl
dH₂O: 6 μl
   10 restriction digest buffer: 2 μl

Restriction digests were then on 1% TAE agarose gel and visualised to identify the presence of NS3 cDNA. The digest products included the pTOPO 2.1 plasmid at ˜3.9 kb and the NS3 insert at ˜1.9 kb.

The sequence fidelity of the NS3 insert was confirmed using the primers shown in Table 1, below:

TABLE 1
Primers used to generate and sequence
the polynucleotide encoding the HCV
NS3 structural protein

| | | | binding | SEQ |
|---|---|---|---|---|
| | Sequence | Tm | position | ID |
| Number | (5′-3′) | (º C.) | in NS3 | NO: |
| | Afl II | | | |
| 107 | gcCTTAAGgcgccca | 70 | 1 | 17 |
| | tcacggcgtacgcc | | | |
| F2 | aaggcggtggac | 40 | 493 | 18 |
| F5 | gcctcactcatatag | 39 | 1613 | 19 |
| | Spe I | | | |
| 108 | gcACTAGTcgtgacga | 68 | 1893 | 20 |
| | cctccaggtcggc | | | |
| 208 | gacatgatcgctggtg | X | * | 21 |
| | ctcac | | | |
| 209 | ccgctgccggtgggag | X | 605 | 22 |
| | cat | | | |
| 210 | gttgtcgtcgtgtcga | X | 1136 | 23 |
| | cc | | | |

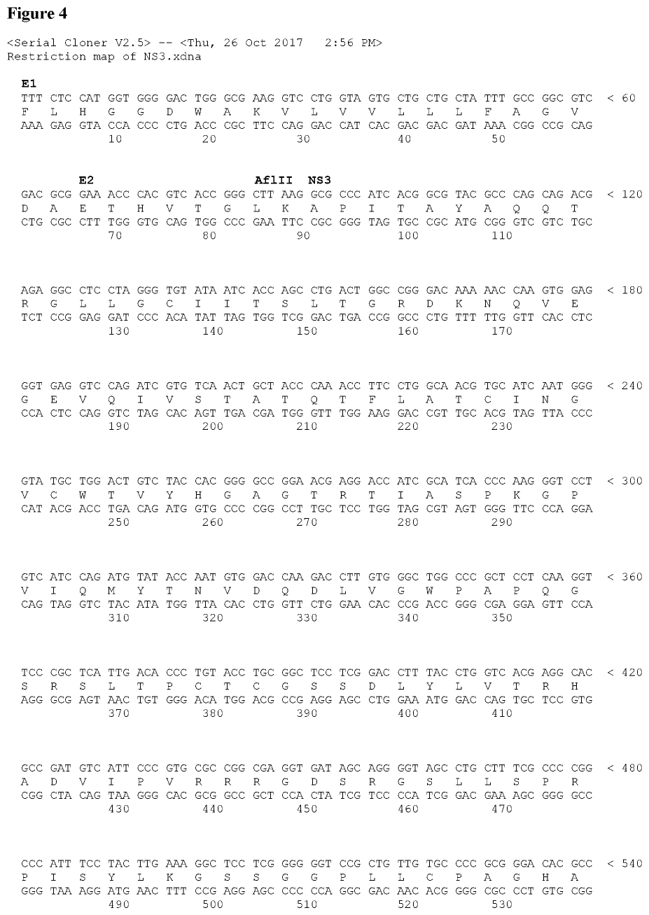

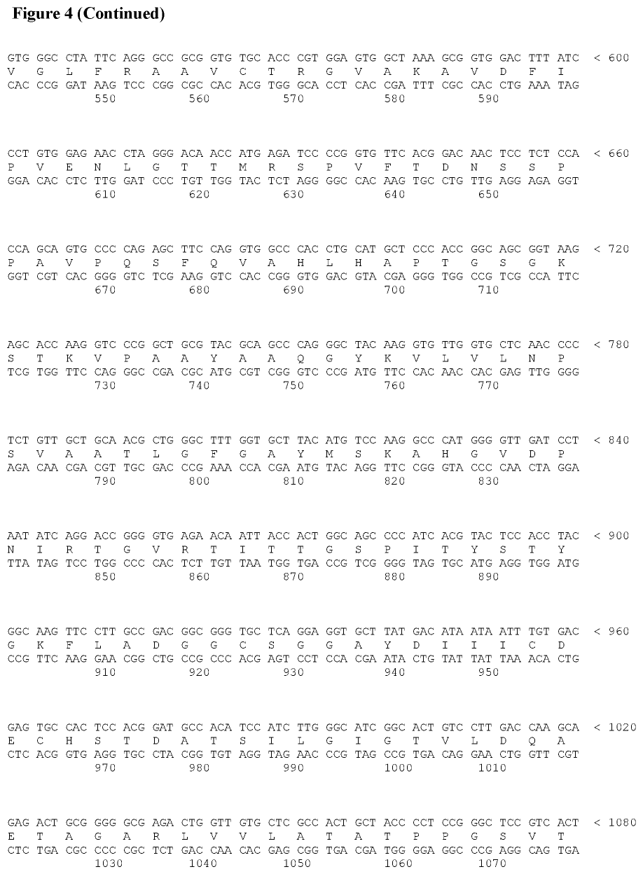

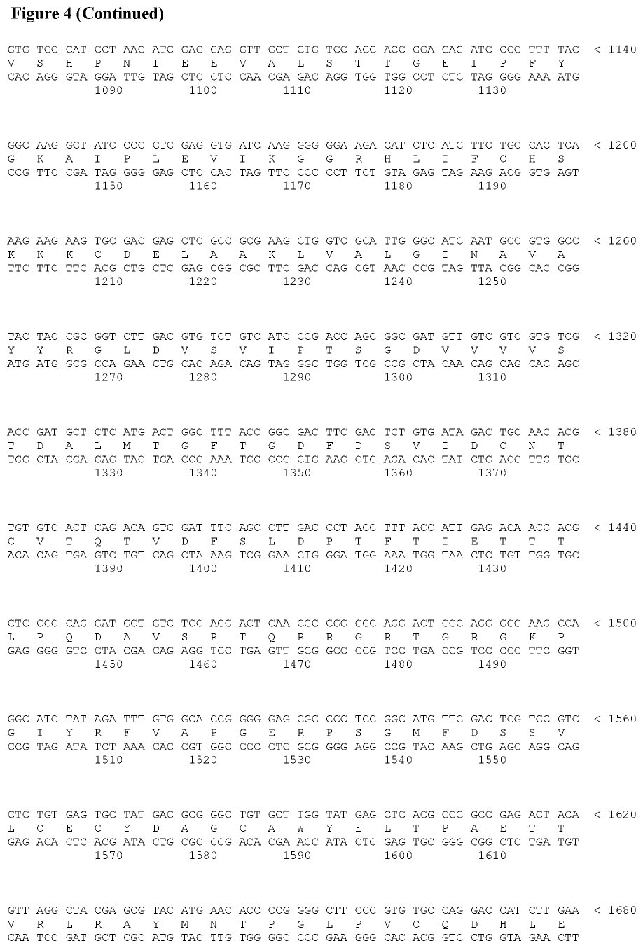

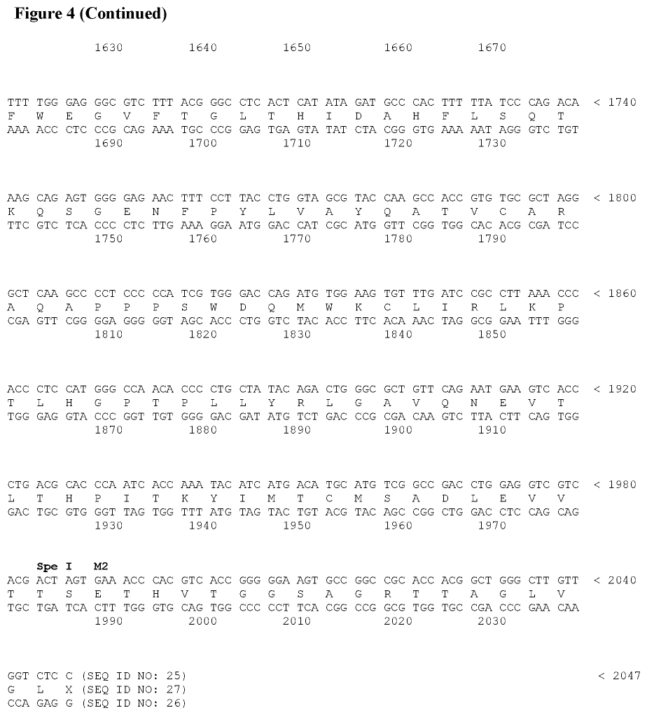

Primer 208 anneals to the nucleic sequence encoding the HCV E1 structural protein and was used to sequence downstream into the newly inserted NS3. The following reagents and conditions were used to amplify the NS3 insert:
Plasmid DNA: 5 μl (800 ng)
Primer: 2 μl (5 μM)
dH₂O: 5 μl
The complete NS3 sequence is shown in FIG. 4.

The purified NS3 cDNA insert was then subcloned into pAdTHCV1aAflII/SpeI plasmid using the following reagents and conditions:
Purified NS3 PCR product: 2 μl
AflII/SpeI digested pAdHCV1aCE1E2 vector: 0.5 μl
ATP: 1 μl
dH₂O: 5.5 μl
T4 DNA Ligase (400 units/μl): 1 μl

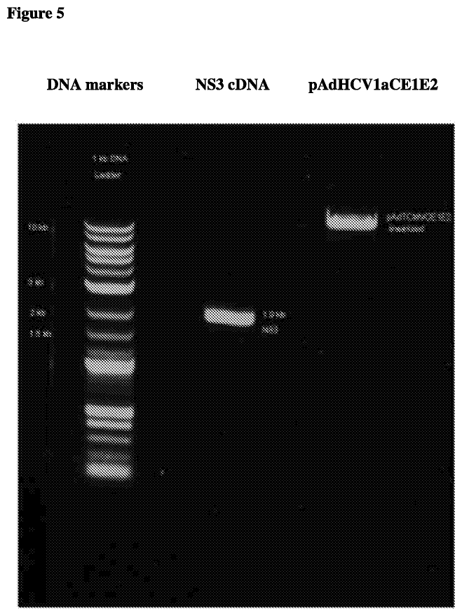

Ligation was performed at 16° C. for 2 hours (see FIG. 5). The ligation product was then transform into chemically competent TOP10F′ cells. Cell colonies were then picked and transferred to LB/Amp medium (50 μg/ml final) and grown overnight at 37° C.

The pAdTHCV1aCE1E2 plasmids were extracted and purisfied using QIAprep Spin Miniprep Kit and restriction digestion was performed to confirm the presence of the NS3 cDNA. Briefly, a restriction digest of the pAdtrackCMVE1E2 plasmid was performed with AflII and SpeI using the following reagents and conditions:
Plasmid DNA: 10 μl
AflII-HF: 1 μl
SpeI-HF: 1 μl
dH₂O: 6 μl
   10 CutSmart buffer: 2 μl

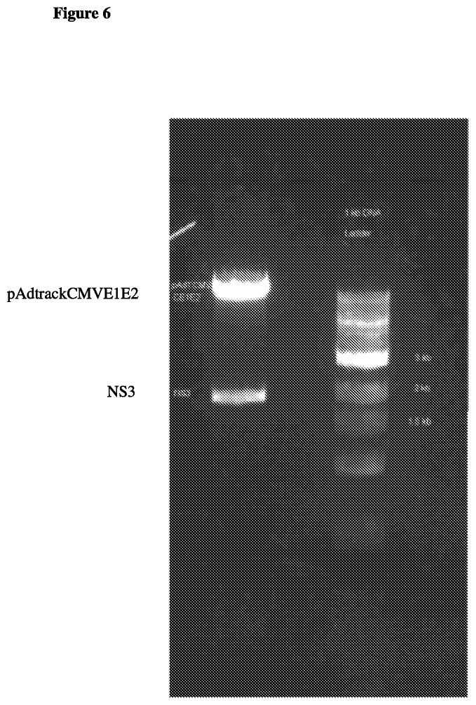

Restriction digests were then run on a 1% TAE agarose gel, and digest products at ˜1.9 kb (NS3 cDNA) and ˜11.5 kb were observed, thereby confirming the presence of the NS3 cDNA in the pAdTHCV1aCE1E2 construct (see FIG. 6).

The pAdTHCV1aCE1NS3E2 clone was then introduced into Adeasier cells. Briefly, the pAdTHCV1aCE1NS3E2 construct was digested with Pme1 for recombination, using the following reagents:
Mini prep DNA: 5 μl
10 x Buffer 4: 2 μl
10 X BSA: 2 μl
PmeI: 1 μl
dH₂O: 10 μl

The digestion products were then loaded onto a 1% agarose gel, and the linearized band purified using a QIAquick Gel Extraction Kit and quantitate on a Nanodrop 2000.

About 1 μg of the PmeI linearized DNA was transformed into Adeasier competent cells by heat shock 42° C. for 45 seconds, and then placed on ice for 2 mins. Following the addition of 1 ml of LB, the cells were shaken for 1 hour at 37° C. before being plated onto LB agar plates containing Kanamycin (50 μg/ml) and incubated overnight at 37° C. The next day, cell colonies were selected and grown in LB with Kanamycin (50 μg/ml) overnight at 37° C. The following day, a QIAprep Spin Miniprep Kit was used to extract plasmids from the cultured cells.

Digestion was then performed on each DNA clone using Pac1 enzyme, as follows:
DNA: 5 μl
10 x buffer: 2 μl
PacI: 2 μl
dH2O: 11 μl

The reagent mixture was incubated for at least an hour at 37° C. and the digest products run on a 1% agarose gel. Positive clones Adeasier1aCE1NS3E2 yielded a large fragment (near 30 kb), plus a smaller fragment of either 3.0 kb or 4.5 kb. Positive clones were selected and re-transformed into Top 10F′ cells and grown up as large cultures. Large-scale production of highly purified, transfection grade DNA was performed using a Qiagen Maxiprep kit.

A PacI digested Maxi-prep Ad1aCE1NS3E2 DNA was then used to transfect 293T cells (E1-transformed human embryonic kidney cells seeded into 2×10 cm² dishes) using Effectene Transfection Reagent, according to the manufacturer's instructions. The progress of the transfection was monitored by fluorescence microscope. Once the cells started to lift, both the cells and media were collected in a 50 ml tubes, spun down on a benchtop centrifuge and then resuspended in 2.0 ml DMEM containing 10% Foetal Calf Serum (FCS). These cells were then used to prepare the primary viral stocks by freeze/thawing. Briefly, resuspended cells were frozen in liquid nitrogen and then thawed in a 37° C. water bath while being vortexed vigorously. The freeze/thaw/vortex cycle was repeated for an additional 3 cycles (four cycles total). Samples were spun briefly and viral supernatant was stored at −20° C.

### Amplification of Virus

Two T-75 flasks of 293T cells (50% to 70% confluent) were infected with ˜500 μl of the above viral T1 stock (for each flask). Any cytopathic effects of the viral infection became evident at 2 to 3 days post infection. Infected cells were collected and subjected to the freeze/thaw/vortex cycles as described above. Viral supernatants were stored at −20° C. (passage 1). The process was repeated until passage 5 (P5). After passage 2, subsequent infections were performed in 293T cells seeded in 175 cm² flasks.

Having determined the effective titre of the NS3 P5 viral stock in Huh7 cells (hepatocyte derived cellular carcinoma cell line), VLP production was scaled-up. To ensure high infection efficiency, cell factories (surface area of ˜1.272 cm²) seeded at 60% confluency were infected with P5 rAdHCV1aCE1NS3E2 amplified viral stock. Cell factories were harvested at 96 hours post-infection in 7 ml of cell lysis buffer (50 mM Tris. HCl pH7.5, 50 mM NaCl and 0.5 mM EDTA) and cells disrupted using a Polytron homogenizer. Cell lysates were clarified by centrifugation, as described previously (Chua et al., PLoS One, 2012;_7(10): e47492), layered on to a 30% sucrose cushion in 20 mM Tris pH7.4 and 150 mM NaCl and centrifuged at 43,800 g for 4 h at 4° C. The pellet was resuspended in 20 mM Tris pH7.4 and 100 mM NaCl and homogenised again with a Dounce homogenizer. Lysates were clarified by centrifugation and then layered on to a 10-40% continuous iodixanol gradient and centrifuged at 16° C. for 14 h at 143,000 g in an SW38 rotor followed by the collection of 12 fractions.

Fractions containing HCV1aCE1NS3E2 VLPs were identified by Western immunoblot with Virostat HCVNS3 antibodies. These localised to Fractions 7-9. These fractions were pooled and concentrated in a Stirred Cell ultrafiltration chamber pressurised with Nitrogen gas using an Ultracel 30 kDa ultrafiltration disc. Briefly, ˜18 ml of pooled iodixanol gradient Fractions 7-9 were diluted to 50 ml with sterile PBS and transferred to the chamber. The chamber was pressurized with nitrogen gas to 55 psi (3.7 kg/cm²) and the VLPs concentrated 10-fold with constant stirring. Purified HCVNS3 VLP were quantitated by Bradford assay. A typical yield of VLP from a single cell factory ranged from 4.75 to 8 mg.

### Example 2—Immunization of Mice

To determine the immunogenicity of the self-cleaving HCV1aCE1NS3E2 VLP vaccine construct, C57BL/6 mice are immunised with two doses of vaccine, 2 weeks apart. Mice are bled by tail vein bleed immediately before the booster dose. One week after the booster, mice are killed and blood, spleen, lymph nodes and liver harvested for analyses.

Anti-HCV antibody titres are determined by ELISA using the HCV1aCE1NS3E2 VLP as the coating antigen. Briefly, 96-well flexible, flat-bottomed polyvinyl chloride (PVC) microtiter plates (Nunc) are coated with 50 μl of 5 μg/ml of HCV1aCE1NS3E2 VLP per well in Carbonate Coating buffer (100 mM Na₂CO₃ and NaHCO₃). Plates are incubated at 4° C. overnight in a humidified chamber. On the second day, the coating solution is discarded, and blocked with 10% BSA (Bovine Serum Albumin) in PBS (Phosphate Buffered Saline) for at least 2 hours at room temperature in a humidified chamber. The plates are then washed 4 times with PBS and blotted dry. 50 μl of anti-HCV antibodies in 5% BSA/PBS at 100 μg/ml is added to each well. The plates are incubated at 4° C. overnight in a humidified chamber. On Day 3, the plates are washed four times with PBS and blotted dry. 50 μl/well of anti-human antibody conjugated to horse radish peroxidase (HRP) (Dako, cat #P0161) diluted 1:500 in 5% BSA/PBS is added to each well. The plates are incubated for 1-2 hours at room temperature in a humidified chamber. The plates are then washed four times in PBS. TMB (tetramethylbenzidine) substrate is tested with conjugate prior to use by mixing 2 drops of both reagents in a counting chamber and observing a colour change. 50 μl of substrate is added to each well and incubated for 10-15 mins at room temperature to observe a colour change. The colourmetric reaction is terminated by adding 50 μl/well of 0.16 M H₂SO₄. The plate is determined for absorbance on a plate reader using a wavelength of 450 nm.

HCV neutralising antibody responses can also be determined by an in vitro neutralisation assay using immune sera from mice and the HCV virus.

NKT and CD4+ and CD8+ T cell responses can be determined in lymphocytes isolated from spleens, lymph nodes and livers of vaccinated mice, using methods that will be familiar to persons skilled in the art.

To determine the frequency of HCV-specific memory CD8+ T cells in the spleen of immunized mice, two approaches can be applied: (1) Elispot to measure the frequency of CD8+ T cells that respond to VLP mixed with antigen presenting cells and the target proteins and peptides derived from these VLP; and (2) flow cytometric analysis to determine the memory cell phenotype (central memory (Tcm), effector memory (Tem)) following immunization.

ELISA and ELISPOT analyses will demonstrate that administration of HCV1aCE1NS3E2 VLP in mice results in an elevation of anti-HCV structural protein antibody titres, B cells and NKT cells, when compared to control (vehicle alone).

The vaccine constructs disclosed herein represent a platform technology that is advantageously adaptable to the production of any VLP-based vaccine for raising an immune response against a target antigen that can be targeted by a humoral and/or cellular immune response, such as viral and cancer-associated antigens. This platform technology is suitably adaptable to the production of VLP of any envelope and non-envelope virus, illustrative examples of which include coronavirus, dengue, hepatitis C and zika viruses.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A vaccine construct comprising a nucleic acid sequence encoding a polyprotein, wherein the polyprotein comprises (i) an immunogen and (ii) two or more viral structural proteins capable of forming a virus-like particle (VLP), wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the liberated structural proteins to self-assemble into a VLP.

2. The vaccine construct according to claim 1, wherein each of the two or more viral structural proteins are separated by a signal peptidase sequence.

3. The vaccine construct according to claim 1, wherein the signal peptidase sequence is capable of being cleaved by a peptidase that is heterologous to the host cell.

4. The vaccine construct according to any one of claim 1, wherein signal peptidase sequence is a signal peptidase sequence utilized by hepatitis C virus, or a cleavable variant thereof.

5. The vaccine construct according to claim 4, wherein the signal peptidase sequence comprises an amino acid sequence selected from the group consisting of SEQ ID NOs: 1-8, and amino acid sequences having at least 80% sequence identity to any of the foregoing.

6-11. (canceled)

12. The vaccine construct according to claim 1, wherein the virus is a Hepacivirus.

13. (canceled)

14. The vaccine construct according to claim 1, wherein the two or more viral structural proteins are selected from the group consisting of an HCV core protein, a HCV envelope glycoprotein E1 and a HCV envelope glycoprotein E2.

15-24. (canceled)

25. The vaccine construct according to claim 1, wherein the immunogen is a heterologous immunogen.

26. (canceled)

27. The vaccine construct according to claim 1, wherein the immunogen is a non-viral antigen.

28. The vaccine construct according to claim 27, wherein the heterologous non-viral antigen is a cancer-associated antigen.

29. A method of producing a VLP, the method comprising:
(i) introducing the vaccine construct according to claim 1 into a host cell; and
(ii) culturing the host cell of (i) under conditions and for a period of time sufficient for the host cell to produce the VLP.

30-31. (canceled)

32. A vaccine composition comprising the vaccine construct according to claim 1.

33.-34. (Canceled)

35. A method of raising an immune response to an immunogen, the method comprising administering to a subject in need thereof the vaccine construct according to claim 1, or the composition according to claim 32.

36-37. (canceled)

38. A kit comprising the vaccine construct according to claim 1.

39. A system or composition comprising (i) a first construct comprising a nucleic acid sequence encoding an immunogen and (ii) a second construct comprising a nucleic acid sequence encoding a polyprotein, wherein the polyprotein comprises two or more viral structural proteins capable of forming a virus-like particle (VLP), wherein at least two of the two or more viral structural proteins are separated by a signal peptidase sequence such that, when the polyprotein is expressed in a host cell, the signal peptidase sequence undergoes host cell peptidase-dependent cleavage to liberate the two or more viral structural proteins, thereby allowing the immunogen and the liberated structural proteins to self-assemble into a VLP.

40. The system or composition according to claim 39, wherein each of the two or more viral structural proteins are separated by a signal peptidase sequence.

41. The system or composition according to claim 39, wherein the signal peptidase sequence is capable of being cleaved by a peptidase that is heterologous to the host cell.

42. The system or composition according to claim 39, wherein signal peptidase sequence is a signal peptidase sequence utilized by hepatitis C virus, or a cleavable variant thereof.

43. The system or composition according to claim 42, wherein the signal peptidase sequence comprises an amino acid sequence selected from the group consisting of SEQ ID NOs:1-8, and amino acid sequences having at least 80% sequence identity to any of the foregoing.

44. (canceled)

45. A method of producing a VLP, the method comprising:
(i) introducing the first and second vaccine constructs according to claim 39 into a host cell; and
(ii) culturing the host cell of (i) under conditions and for a period of time sufficient for the host cell to produce the VLP.

46-47. (canceled)

48. A kit comprising the composition according to claim 32.


SEQUENCE LISTING (40 sequences)

SEQ ID NO: 1 - protein, 21 aa, Hepacivirus C
  source 1..21: note = Core-E1 signal peptidase - HCV genotype 1a
GCSFSIFLLALLSCLTVPASA

SEQ ID NO: 2 - protein, 21 aa, Hepacivirus C
  source 1..21: note = Core-E1 signal peptidase - HCV genotype 1b
GCSFSIFLLALLSCLTIPASA

SEQ ID NO: 3 - protein, 21 aa, Hepacivirus C
  source 1..21: note = Core-E1 signal peptidase - HCV genotype 2a
GFPFSIFLLALLSCITVPVSA

SEQ ID NO: 4 - protein, 21 aa, Hepacivirus C
  source 1..21: note = Core-E1 signal peptidase - HCV genotype 3a
GCSFSVFLLALFSCLIHPAAS

SEQ ID NO: 5 - protein, 21 aa, Hepacivirus C
  source 1..21: note = Core-E1 signal peptidase - HCV genotype 4a
GCSFSIFLLALLSCLTVPASA

SEQ ID NO: 6 - protein, 21 aa, Hepacivirus C
  source 1..21: note = Core-E1 signal peptidase - HCV genotype 5a
GCSFSIFILALLSCLTVPTSA

SEQ ID NO: 7 - protein, 21 aa, Hepacivirus C
  source 1..21: note = Core-E1 signal peptidase - HCV genotype 6a
GCSFSIFLLALLSCLTTPASA

SEQ ID NO: 8 - protein, 21 aa, Hepacivirus C
  source 1..21: note = Core-E1 signal peptidase - HCV genotype 7a
GCSFSIFLLALLSCLTVPASA

SEQ ID NO: 9 - protein, 18 aa, Measles morbillivirus
  source 1..18: note = Measles Virus Fusion Protein
KLLSLIKGVIVHRLEGVE

SEQ ID NO: 10 - protein, 19 aa, Clostridium tetani
  source 1..19: note = Tetanus Toxin
NSVDDALINSTIYSYFPSV

SEQ ID NO: 11 - protein, 17 aa, Clostridium tetani
  source 1..17: note = Tetanus Toxin
PGINGKAIHLVNNQSSE

SEQ ID NO: 12 - protein, 15 aa, Clostridium tetani
  source 1..15: note = Tetanus Toxin P2
QYIKANSKFIGITEL

SEQ ID NO: 13 - protein, 21 aa, Clostridium tetani
  source 1..21: note = Tetanus Toxin P30
FNNFTVSFWLRVPKVSASHLE

SEQ ID NO: 14 - protein, 15 aa, Measles morbillivirus
  source 1..15: note = Measles Virus Fusion Protein
LSEIKGVIVHRLEGV

SEQ ID NO: 15 - protein, 15 aa, Hepatitis B virus
FFLLTRILTIPQSLN

SEQ ID NO: 16 - protein, 21 aa, Plasmodium sp.
  source 1..21: note = circumsporozoite protein
TCGVGVRVRSRVNAANKKPEL

SEQ ID NO: 17 - DNA, 29 bp, synthetic construct
  source 1..29: mol_type = other DNA
gccttaaggcgcccatcacggcgtacgcc

SEQ ID NO: 18 - DNA, 12 bp, synthetic construct
  source 1..12: mol_type = other DNA
aaggcggtggac

SEQ ID NO: 19 - DNA, 15 bp, synthetic construct
  source 1..15: mol_type = other DNA
gcctcactcatatag

SEQ ID NO: 20 - DNA, 29 bp, synthetic construct
  source 1..29: mol_type = other DNA
gcactagtcgtgacgacctccaggtcggc

SEQ ID NO: 21 - DNA, 21 bp, synthetic construct
  source 1..21: mol_type = other DNA
gacatgatcgctggtgctcac

SEQ ID NO: 22 - DNA, 19 bp, synthetic construct
  source 1..19: mol_type = other DNA
ccgctgccggtgggagcat

SEQ ID NO: 23 - DNA, 18 bp, synthetic construct
  source 1..18: mol_type = other DNA
gttgtcgtcgtgtcgacc

SEQ ID NO: 24 - DNA, 1893 bp, Hepacivirus C
  source 1..1893: mol_type = genomic DNA; note = HCV1a clone H77 NS3 nucleotide sequence
gcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgcacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaggcggtggactttatccctgtggagaacctagagacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccctttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctacagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttctatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacg

SEQ ID NO: 25 - DNA, 2047 bp, synthetic construct
  source 1..2047: mol_type = other DNA
tttctccatggtggggactgggcgaaggtcctggtagtgctgctgctatttgccggcgtcgacgcggaaacccacgtcaccgggcttaaggcgcccatcacggcgtacgcccagcagacgagaggcctcctagggtgtataatcaccagcctgactggccgggacaaaaaccaagtggagggtgaggtccagatcgtgtcaactgctacccaaaccttcctggcaacgtgcatcaatggggtatgctggactgtctaccacggggccggaacgaggaccatcgcatcacccaagggtcctgtcatccagatgtataccaatgtggaccaagaccttgtgggctggcccgctcctcaaggttcccgctcattgacaccctgtacctgcggctcctcggacctttacctggtcacgaggcacgccgatgtcattcccgtgcgccggcgaggtgatagcaggggtagcctgctttcgccccggcccatttcctacttgaaaggctcctcggggggtccgctgttgtgccccgcgggacacgccgtgggcctattcagggccgcggtgtgcacccgtggagtggctaaagcggtggactttatccctgtggagaacctagggacaaccatgagatccccggtgttcacggacaactcctctccaccagcagtgccccagagcttccaggtggcccacctgcatgctcccaccggcagcggtaagagcaccaaggtcccggctgcgtacgcagcccagggctacaaggtgttggtgctcaacccctctgttgctgcaacgctgggctttggtgcttacatgtccaaggcccatggggttgatcctaatatcaggaccggggtgagaacaattaccactggcagccccatcacgtactccacctacggcaagttccttgccgacggcgggtgctcaggaggtgcttatgacataataatttgtgacgagtgccactccacggatgccacatccatcttgggcatcggcactgtccttgaccaagcagagactgcgggggcgagactggttgtgctcgccactgctacccctccgggctccgtcactgtgtcccatcctaacatcgaggaggttgctctgtccaccaccggagagatccccttttacggcaaggctatccccctcgaggtgatcaaggggggaagacatctcatcttctgccactcaaagaagaagtgcgacgagctcgccgcgaagctggtcgcattgggcatcaatgccgtggcctactaccgcggtcttgacgtgtctgtcatcccgaccagcggcgatgttgtcgtcgtgtcgaccgatgctctcatgactggctttaccggcgacttcgactctgtgatagactgcaacacgtgtgtcactcagacagtcgatttcagccttgaccctacctttaccattgagacaaccacgctcccccaggatgctgtctccaggactcaacgccggggcaggactggcagggggaagccaggcatctatagatttgtggcaccgggggagcgcccctccggcatgttcgactcgtccgtcctctgtgagtgctatgacgcgggctgtgcttggtatgagctcacgcccgccgagactacagttaggctacgagcgtacatgaacaccccggggcttcccgtgtgccaggaccatcttgaattttgggagggcgtctttacgggcctcactcatatagatgcccactttttatcccagacaaagcagagtggggagaactttccttacctggtagcgtaccaagccaccgtgtgcgctagggctcaagcccctcccccatcgtgggaccagatgtggaagtgtttgatccgccttaaacccaccctccatgggccaacacccctgctatacagactgggcgctgttcagaatgaagtcaccctgacgcacccaatcaccaaatacatcatgacatgcatgtcggccgacctggaggtcgtcacgactagtgaaacccacgtcaccgggggaagtgccggccgcaccacggctgggcttgttggtctcc

SEQ ID NO: 26 - DNA, 2047 bp, synthetic construct
  source 1..2047: mol_type = other DNA
ggagaccaacaagcccagccgtggtgcggccggcacttcccccggtgacgtgggtttcactagtcgtgacgacctccaggtcggccgacatgcatgtcatgatgtatttggtgattgggtgcgtcagggtgacttcattctgaacagcgcccagtctgtatagcaggggtgttggcccatggagggtgggtttaaggcggatcaaacacttccacatctggtcccacgatgggggaggggcttgagccctagcgcacacggtggcttggtacgctaccaggtaaggaaagttctccccactctgctttgtctgggataaaaagtgggcatctatatgagtgaggcccgtaaagacgccctcccaaaattcaagatggtcctggcacacgggaagccccggggtgttcatgtacgctcgtagcctaactgtagtctcggcgggcgtgagctcataccaagcacagcccgcgtcatagcactcacagaggacggacgagtcgaacatgccggaggggcgctcccccggtgccacaaatctatagatgcctggcttccccctgccagtcctgccccggcgttgagtcctggagacagcatcctgggggagcgtggttgtctcaatggtaaaggtagggtcaaggctgaaatcgactgtctgagtgacacacgtgttgcagtctatcacagagtcgaagtcgccggtaaagccagtcatgagagcatcggtcgacacgacgacaacatcgccgctggtcgggatgacagacacgtcaagaccgcggtagtaggccacggcattgatgcccaatgcgaccagcttcgcggcgagctcgtcgcacttcttctttgagtggcagaagatgagatgtcttccccccttgatcacctcgagggggatagccttgccgtaaaaggggatctctccggtggtggacagagcaacctcctcgatgttaggatgggacacagtgacggagcccggaggggtagcagtggcgagcacaaccagtctcgcccccgcagtctctgcttggtcaaggacagtgccgatgcccaagatggatgtggcatccgtggagtggcactcgtcacaaattattatgtcataagcacctcctgagcacccgccgtcggcaaggaacttgccgtaggtggagtacgtgatggggctgccagtggtaattgttctcaccccggtcctgatattaggatcaaccccatgggccttggacatgtaagcaccaaagcccagcgttgcagcaacagaggggttgagcaccaacaccttgtagccctgggctgcgtacgcagccgggaccttggtgctcttaccgctgccggtgggagcatgcaggtgggccacctggaagctctggggcactgctggtggagaggagttgtccgtgaacaccggggatctcatggttgtccctaggttctccacagggataaagtccaccgctttagccactccacgggtgcacaccgcggccctgaataggcccacggcgtgtcccgcggggcacaacagcggaccccccgaggagcctttcaagtaggaaatgggccggggcgaaagcaggctacccctgctatcacctcgccggcgcacgggaatgacatcggcgtgcctcgtgaccaggtaaaggtccgaggagccgcaggtacagggtgtcaatgagcgggaaccttgaggagcgggccagcccacaaggtcttggtccacattggtatacatctggatgacaggacccttgggtgatgcgatggtcctcgttccggccccgtggtagacagtccagcataccccattgatgcacgttgccaggaaggtttgggtagcagttgacacgatctggacctcaccctccacttggtttttgtcccggccagtcaggctggtgattatacaccctaggaggcctctcgtctgctgggcgtacgccgtgatgggcgccttaagcccggtgacgtgggtttccgcgtcgacgccggcaaatagcagcagcactaccaggaccttcgcccagtccccaccatggagaaa

SEQ ID NO: 27 - protein, 683 aa, synthetic construct
FLHGGDWAKVLVVLLLFAGVDAETHVTGLKAPITAYAQQTRGLLGCIITSLTGRDKNQVEGEVQIVSTATQTFLATCINGVCWTVYHGAGTRTIASPKGPVIQMYTNVDQDLVGWPAPQGSRSLTPCTCGSSDLYLVTRHADVIPVRRRGDSRGSLLSPRPISYLKGSSGGPLLCPAGHAVGLFRAAVCTRGVAKAVDFIPVENLGTTMRSPVFTDNSSPPAVPQSFQVAHLHAPTGSGKSTKVPAAYAAQGYKVLVLNPSVAATLGFGAYMSKAHGVDPNIRTGVRTITTGSPITYSTYGKFLADGGCSGGAYDIIICDECHSTDATSILGIGTVLDQAETAGARLVVLATATPPGSVTVSHPNIEEVALSTTGEIPFYGKAIPLEVIKGGRHLIFCHSKKKCDELAAKLVALGINAVAYYRGLDVSVIPTSGDVVVVSTDALMTGFTGDFDSVIDCNTCVTQTVDFSLDPTFTIETTTLPQDAVSRTQRRGRTGRGKPGIYRFVAPGERPSGMFDSSVLCECYDAGCAWYELTPAETTVRLRAYMNTPGLPVCQDHLEFWEGVFTGLTHIDAHFLSQTKQSGENFPYLVAYQATVCARAQAPPPSWDQMWKCLIRLKPTLHGPTPLLYRLGAVQNEVTLTHPITKYIMTCMSADLEVVTTSETHVTGGSAGRTTAGLVGLX

SEQ ID NO: 28 - DNA, 69 bp, synthetic construct
  source 1..69: mol_type = other DNA
gcctctagagccaccatgcatcaccatcaccatcacacaagcacgaatcctaaacctcaaagaaaaacc

SEQ ID NO: 29 - DNA, 35 bp, synthetic construct
  source 1..35: mol_type = other DNA
ggcttaagcccggtgacgtgggtttccgcgtcgac

SEQ ID NO: 30 - DNA, 41 bp, synthetic construct
  source 1..41: mol_type = other DNA
cgactagtgaaacccacgtcaccgggggaagtgccggccgc

SEQ ID NO: 31 - DNA, 46 bp, synthetic construct
  source 1..46: mol_type = other DNA
cggatatctcatcacgcctccgcttgggatatgagtaacatcatcc

SEQ ID NO: 32 - DNA, 38 bp, synthetic construct
  source 1..38: mol_type = other DNA
gccttaagacagtgagcaagcagatcctgaagaacacc

SEQ ID NO: 33 - DNA, 35 bp, synthetic construct
  source 1..35: mol_type = other DNA
gcactagtcacccactcgtgcaggctgcccagggg

SEQ ID NO: 34 - DNA, 3030 bp, synthetic construct
  source 1..3030: mol_type = other DNA
tctagagccaccatgcatcaccatcaccatcactgtagcacgaatcctaaacctcaaagaaaaaccaaacgtaacaccaaccgtcgcccacaggacgtcaagttcccgggtggcggtcagatcgttggtggagtttacttgttgccgcgcaggggccctagattgggtgtgcgcgcgacgaggaagacttccgagcggtcgcaacctcgaggtagacgtcagcctatccccaaggcacgtcggcccgagggcaggacctgggctcagcccgggtacccttggcccctctatggcaatgagggttgcgggtgggcgggatggctcctgtctccccgtggctctcggcctagctggggccccacagacccccggcgtaggtcgcgcaatttgggtaaggtcatcgatacccttacgtgcggcttcgccgacctcatggggtacataccgctcgtcggcgcccctcttggaggcgctgccagggccctggcgcatggcgtccgggttctggaagacggcgtgaactatgcaacagggaaccttcctggttgctctttctctatcttccttctggccctgctctcttgcctgaccgtgcccgcttcagcctaccaagtgcgcaattcctcggggctttaccatgtcaccaatgattgccctaactcgagtattgtgtacgaggcggccgatgccatcctgcacactccggggtgtgtcccttgcgttcgcgagggtaacgcctcgaggtgttgggtggcggtgacccccacggtggccaccagggacggcaaactccccacaacgcagcttcgacgtcatatcgatctgcttgtcgggagcgccaccctctgctcggccctctacgtgggggacctgtgcgggtctgtctttcttgttggtcaactgtttaccttctctcccaggcgccactggacgacgcaagactgcaattgttctatctatcccggccatataacgggtcatcgcatggcatgggatatgatgatgaactggtcccctacggcagcgttggtggtagctcagctgctccggatcccacaagccatcatggacatgatcgctggtgctcactggggagtcctggcgggcatagcgtatttctccatggtggggaactgggcgaaggtcctggtagtgctgctgctatttgccggcgtcgacgcggaaacccacgtcaccgggcttaagtgtgtgagcaagcagatcctgaagaacaccggcctgcaggagatcatgagcttcaaggtgaacctggagggcgtggtgaacaaccacgtgttcaccatggagggctgcggcaagggcaacatcctgttcggcaaccagctggtgcagatccgcgtgaccaagggcgcccccctgcccttcgccttcgacatcctgagccccgccttccagtacggcaaccgcaccttcaccaagtaccccgaggacatcagcgacttcttcatccagagcttccccgccggcttcgtgtacgagcgcaccctgcgctacgaggacggcggcctggtggagatccgcagcgacatcaacctgatcgaggagatgttcgtgtaccgcgtggagtacaagggccgcaacttccccaacgacggccccgtgatgaagaagaccatcaccggcctgcagcccagcttcgaggtggtgtacatgaacgacggcgtgctggtgggccaggtgatcctggtgtaccgcctgaacagcggcaagttctacagctgccacatgcgcaccctgatgaagagcaagggcgtggtgaaggacttccccgagtaccacttcatccagcaccgcctggagaagacctacgtggaggacggcggcttcgtggagcagcacgagaccgccatcgcccagctgaccagcctgggcaagcccctgggcagcctgcacgagtgggtgactagtgaaacccacgtcaccgggggaagtgccggccgcaccacggctgggcttgttggtctccttacaccaggcgccaagcagaacatccaactgatcaacaccaacggcagttggcacatcaatagcacggccttgaactgcaatgaaagccttaacaccggctggttagcagggctcttctatcagcacaaattcaactcttcaggctgtcctgagaggttggccagctgccgacgccttaccgattttgcccagggctggggtcctatcagttatgccaacggaagcggcctcgacgaacgcccctactgctggcactaccctccaagaccttgtggcattgtgcccgcaaagagcgtgtgtggcccggtatattgcttcactcccagccccgtggtggtgggaacgaccgacaggtcgggcgcgcctacctacagctggggtgcaaatgatacggatgtcttcgtccttaacaacaccaggccaccgctgggcaattggttcggttgtacctggatgaactcaactggattcaccaaagtgtgcggagcgcccccttgtgtcatcggaggggtgggcaacaacaccttgctctgccccactgattgtttccgcaagcatccggaagccacatactctcggtgcggctccggtccctggattacacccaggtgcatggtcgactacccgtataggctttggcactatccttgtaccatcaattacaccatattcaaagtcaggatgtacgtgggaggggtcgagcacaggctggaagcggcctgcaactggacgcggggcgaacgctgtgatctggaagacagggacaggtccgagctcagcccattgctgctgtccaccacacagtggcaggtccttccgtgttctttcacgaccctgccagccttgtccaccggcctcatccacctccaccagaacattgtggacgtgcagtacttgtacggggtagggtcaagcatcgcgtcctgggccattaagtgggagtacgtcgttctcctgttcctcctgcttgcagacgcgcgcgtctgctcctgcttgtggatgatgttactcatatcccaagcggaggcgtgatgagatatc

SEQ ID NO: 35 - DNA, 21 bp, synthetic construct
  source 1..21: mol_type = other DNA
gcgcccatcacggcgtacgcc

SEQ ID NO: 36 - DNA, 12 bp, synthetic construct
  source 1..12: mol_type = other DNA
aaggcggtggac

SEQ ID NO: 37 - DNA, 19 bp, synthetic construct
  source 1..19: mol_type = other DNA
tacgagggtggccgtcgcc

SEQ ID NO: 38 - DNA, 18 bp, synthetic construct
  source 1..18: mol_type = other DNA
gttgtcgtcgtgtcgacc

SEQ ID NO: 39 - DNA, 15 bp, synthetic construct
  source 1..15: mol_type = other DNA
gcctcactcatatag

SEQ ID NO: 40 - DNA, 21 bp, synthetic construct
  source 1..21: mol_type = other DNA
cggctggacctccagcagtgc